(19) United States
(12) Patent Application Publication
(10) Pub. No.: US 2023/0003715 A1
(43) Pub. Date: Jan. 5, 2023

(54) AZAPODOPHYLLOTOXIN DERIVATIVES AND METHODS OF TREATING LYMPHOMA AND KIDNEY CANCER

(71) Applicant: The Board of Trustees of the Leland Stanford Junior University, Stanford, CA (US)

(72) Inventors: Arvin Gouw, San Francisco, CA (US); Sanjay Malhotra, Palo Alto, CA (US); Dean W. Felsher, San Mateo, CA (US)

(21) Appl. No.: 17/772,940

(22) PCT Filed: Nov. 3, 2020
(86) PCT No.: PCT/US2020/058666

§ 371 Date: Apr. 28, 2022

Related U.S. Application Data
(60) Provisional application No. 62/930,346, filed on Nov. 4, 2019.

(51) Int. Cl. G01N 33/50; A61K 31/473; A61K 31/4741; A61P 35/00; G01N 33/574

(52) U.S. Cl. CPC G01N 33/5011; A61K 31/473; A61K 31/4741; A61P 35/00; G01N 33/57484

(57) ABSTRACT

Methods of inhibiting the proliferation of a cancer cell, and treating cancer in an individual are provided. Aspects of the subject methods include contacting a cancer cell with an azapodophyllotoxin derivative, where the contacting is effective to inhibit tubulin polymerization and monoglycerol metabolism to inhibit proliferation of cancer in the cell. In certain cases, the cancer cell is a renal cancer cell (RCC) or a lymphoma cell. Aspects of the methods also include administering to a subject an effective amount of an azapodophyllotoxin derivative to treat the subject for cancer, where the cancer is selected from renal cancer and lymphoma. Also provided is a method of monitoring tumor regression in an individual, and methods of identifying a cancer suppressing compound.

### CROSS REFERENCE TO RELATED APPLICATION

This application claims priority to U.S. Provisional Application No. 62/930,346 filed Nov. 4, 2019, which is incorporated herein in its entirety for all purpose.

### INTRODUCTION

Lymphoma is one of the most common types of cancer, regardless of age. Kidney cancer is also one of the top most prevalent cancer types. However, currently, there is no cure for kidney cancer and only a handful of known cures for specific types of lymphomas (Rossi et al., World Journal of Urology, 2018; 36(9):1341-53; and O'Hare et al., Blood, 2007; 110(7):2242-9).

Microtubules are cytoskeletal protein polymers composed of α and β tubulin heterodimers. They are involved in essential cellular processes such as migration, intracellular transport and mitosis (Wade, Molecular biotechnology, 2009; 43(2):177-91; Bates et al., British journal of clinical pharmacology, 2017; 83(2):255-68; Jordan et al., Nature reviews Cancer. 2004; 4(4):253-65; Checchi et al., Trends in pharmacological sciences, 2003; 24(7):361-5). Inhibition of microtubule polymerization leads to disruption of mitotic spindle formation, blocks mitosis and arrests the cell cycle in the G2/M phase, leading to apoptosis. Thus, tubulin is considered as an important target for anticancer drug development. Most of the drugs that inhibit microtubule assembly e.g. paclitaxel (taxane), vinblastine and vincristine (vinca alkaloids) and podophyllotoxin (lignans) are derived from natural products (Negi et al., Bioorganic & medicinal chemistry, 2015; 23(3):373-89; Steinmetz et al., Trends in cell biology, 2018; 28(10):776-92; Schiff et al., Nature, 1979; 277(5698):665-7). These drugs, despite their significant clinical relevance, possess serious problems in terms of pharmacokinetics, toxicity and resistance, which limits their therapeutic potential(Kavallaris, Nature reviews Cancer, 2010; 10(3):194-204; Sparreboom et al., Proceedings of the National Academy of Sciences of the United States of America, 1997; 94(5):2031-5; Bates et al., Clinical cancer research: an official journal of the American Association for Cancer Research, 2004; 10(14):4724-33). Also, due to presence of complex ring systems and multiple chiral centers their structures are very complex which require difficult and laborious synthetic steps. This makes large-scale supply of these compounds challenging and impedes lead optimization process. Thus, there is a great interest in the development of new and structurally simple microtubule-binding anticancer agents that can be synthesized easily to overcome these limitations. Development of libraries of new microtubule-binding anticancer agents would lead to more informative SAR studies and expeditious structure optimization.

### SUMMARY

Methods of inhibiting the proliferation of a cancer cell, and treating cancer in an individual are provided. Aspects of the subject methods include contacting a cancer cell with an azapodophyllotoxin derivative, where the contacting is effective to inhibit tubulin polymerization and monoglycerol metabolism to inhibit proliferation of cancer in the cell. In certain cases, the cancer cell is a renal cancer cell (RCC) or a lymphoma cell. Aspects of the methods include administering to a subject an effective amount of an azapodophyllotoxin derivative to treat the subject for cancer, where the cancer is selected from renal cancer and lymphoma. In certain cases the administering is effective to inhibit tubulin polymerization and monoglycerol metabolism. Also provided is a method of monitoring tumor regression in an individual. The method of monitoring tumor regression includes assaying, in a sample obtained from the individual during a treatment regime for cancer, changes in tubulin protein levels, wherein a level of tubulin protein that is lower than a pretreatment level of tubulin indicates tumor regression. Also provided are methods of identifying a cancer suppressing compound. Aspects of the methods include contacting a cancer cell with a candidate compound; determining if tubulin protein levels are decreased relative to the cancer cell in the absence of the candidate compound; and determining if monoglycerol levels are increased relative to the cancer cell in the absence of the candidate compound, wherein a decrease in tubulin protein level and an increase in levels of monoglycerols identifies the candidate compound as a cancer suppressing compound. In some cases, the cancer is selected from renal cancer or lymphoma.

These and other advantages and features of the disclosure will become apparent to those persons skilled in the art upon reading the details of the compositions and methods of use, which are more fully described below.

### BRIEF DESCRIPTION OF THE FIGURES

The invention is best understood from the following detailed description when read in conjunction with the accompanying figures. It is emphasized that, according to common practice, the various features of the figures are not to-scale. On the contrary, the dimensions of the various features are arbitrarily expanded or reduced for clarity. Included in the drawings are the following figures. It is understood that the figures, described below, are for illustration purposes only. The figures are not intended to limit the scope of the present teachings in any way.

FIG. 1 depicts exemplary azapodophyllotoxin (AZP) derivatives.

FIG. 2 illustrates treatment of murine renal cancer cell (RCC) E28 cells with exemplary AZP derivatives decreases cell proliferation in 48 hours. From left to right, treatment of RCC E28 cells is depicted with DMSO and corresponding AZP derivatives at concentrations of 70 nM, 140 nM and 280 nM for 48 hours.

FIG. 3 illustrates that exemplary AZP derivatives decrease human A498 RCC proliferation in 48 hours. From left to right: control, AR-02, AR-03, AR-038, AR-051, AR-061, AR-065 and NSC750212 efficacy in suppression of proliferation in vitro in human RCC A498 cell line over the course of 48 hours.

FIG. 4 illustrates that NSC750212 suppresses proliferation of lymphoma lines. From left to right: human lymphoma lines CCRF, DND-41, KOPT and MYC-driven lymphoma lines P493-6 and 6780 proliferative responses to 70 nM, 140 nM, or 280 nM dosage of NSC750212.

FIG. 5, panels A-C illustrates a comparison of xenograft volume between DMSO-treated (control) group and NSC750212-treated group upon termination of treatment in human RCC xenografts.

FIG. 6 illustrates a comparison of xenograft volume between DMSO-treated (control) group and NSC750212-treated group upon termination of treatment in human lymphoma xenografts.

FIG. 7, panels A-D illustrates that exemplary compound NSC750212 inhibits tubulin polymerization. Panel A, top curve shows paclitaxel as a tubulin polymerization stabilizer (negative control), middle curve shows nocodzole as a tubulin polymerization destabilizer (positive control), and bottom curve shows NSC750212 exhibits an even higher efficacy that the positive control. Panel B, from left to right: control and NSC750212 fine needle aspirations, and control and NSC750212 core biopsies from mouse tumors taken during the course of treatment. Panel C, illustrates NIA data shown in gel display (e.g., like Western Blot). From left to right: control and NSC750212 treated ex vivo fine needle aspirates, and control and NSC750212-treated ex vivo core biopsies. Panel D, shows in vivo control tumor sample (dark grey curve) vs in vivo NSC750212-treated tumor sample (light grey curve). Chemiluminescence (y-axis) is used to convey the level of expression of tubulin in the sample, and the area under the highest peak is quantifiable and can be sued to compare tubulin levels between control and drug treated samples objectively.

FIG. 8 illustrates comparison of DESI-MSI profiles of monoglycerols vs fatty acids in normal vs NSC750212-treated group upon termination of treatment in primary MYC-driven RCC.

### DEFINITIONS

Before embodiments of the present disclosure are further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Any methods and materials similar or equivalent to those described herein can also be used in the practice or testing of embodiments of the present disclosure.

It must be noted that as used herein and in the appended claims, the singular forms “a”, “and”, and “the” include plural referents unless the context clearly dictates otherwise. Thus, for example, reference to “a compound” includes not only a single compound but also a combination of two or more compounds, reference to “a substituent” includes a single substituent as well as two or more substituents, and the like.

In describing and claiming the present invention, certain terminology will be used in accordance with the definitions set out below. It will be appreciated that the definitions provided herein are not intended to be mutually exclusive. Accordingly, some chemical moieties may fall within the definition of more than one term.

As used herein, the phrases “for example,” “for instance,” “such as,” or “including” are meant to introduce examples that further clarify more general subject matter. These examples are provided only as an aid for understanding the disclosure, and are not meant to be limiting in any fashion.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “active agent,” “antagonist”, “inhibitor”, “drug” and “pharmacologically active agent” are used interchangeably herein to refer to a chemical material or compound which, when administered to an organism (human or animal) induces a desired pharmacologic and/or physiologic effect by local and/or systemic action.

As used herein, the terms “treatment,” “treating,” and the like, refer to obtaining a desired pharmacologic and/or physiologic effect, such as reduction of tumor burden. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease (e.g., reduction of tumor burden).

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

The term “alkyl” as used herein refers to a branched or unbranched saturated hydrocarbon group (i.e., a mono-radical) typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although not necessarily, alkyl groups herein may contain 1 to about 18 carbon atoms, and such groups may contain 1 to about 12 carbon atoms. The term “lower alkyl” intends an alkyl group of 1 to 6 carbon atoms. “Substituted alkyl” refers to alkyl substituted with one or more substituent groups, and this includes instances wherein two hydrogen atoms from the same carbon atom in an alkyl substituent are replaced, such as in a carbonyl group (i.e., a substituted alkyl group may include a —C(═O)— moiety). The terms “heteroatom-containing alkyl” and “heteroalkyl” refer to an alkyl substituent in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “substituted alkyl” is meant to include an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein may contain 2 to about 18 carbon atoms, and for example may contain 2 to 12 carbon atoms. The term “lower alkenyl” intends an alkenyl group of 2 to 6 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C1-C6 alkoxy” or “lower alkoxy” herein may, for example, may contain 1 to 3 carbon atoms, and as a further example, such substituents may contain 1 or 2 carbon atoms (i.e., methoxy and ethoxy).

The term “substituted alkoxy” refers to the groups substituted alkyl-O—, substituted alkenyl-O—, substituted cycloalkyl-O—, substituted cycloalkenyl-O—, and substituted alkynyl-O-where substituted alkyl, substituted alkenyl, substituted cycloalkyl, substituted cycloalkenyl and substituted alkynyl are as defined herein.

As used herein, “carbocycle” or “carbocyclic ring” is intended to mean any stable monocyclic, bicyclic, or tricyclic ring having the specified number of carbons, any of which may be saturated, unsaturated, or aromatic. For example a C3-14 carbocycle is intended to mean a mono-, bi-, or tricyclic ring having 3, 4, 5, 6, 7, 8, 9, 10, 11, 12, 13, or 14 carbon atoms.

Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when a covalent bond or one or more carbon atoms link two non-adjacent carbon atoms in a ring. In one embodiment, bridge rings are one or two carbon atoms. It is noted that a bridge always converts a monocyclic ring into a bicyclic ring. When a ring is bridged, the substituents recited for the ring may also be present on the bridge. Fused (e.g., naphthyl and tetrahydronaphthyl) and spiro rings are also included.

The term “aryl” as used herein, and unless otherwise specified, refers to an aromatic substituent generally, although not necessarily, containing 5 to 30 carbon atoms and containing a single aromatic ring or multiple aromatic rings that are fused together, directly linked, or indirectly linked (such that the different aromatic rings are bound to a common group such as a methylene or ethylene moiety). Aryl groups may, for example, contain 5 to 20 carbon atoms, and as a further example, aryl groups may contain 5 to 12 carbon atoms. For example, aryl groups may contain one aromatic ring or two or more fused or linked aromatic rings (i.e., biaryl, aryl-substituted aryl, etc.). Examples include phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. Aryl is intended to include stable cyclic, heterocyclic, polycyclic, and polyheterocyclic unsaturated C₃-C₁₄ moieties, exemplified but not limited to phenyl, biphenyl, naphthyl, pyridyl, furyl, thiophenyl, imidazoyl, pyrimidinyl, and oxazoyl; which may further be substituted with one to five members selected from hydroxy, C₁-C₈ alkoxy, C₁-C₈ branched or straight-chain alkyl, acyloxy, carbamoyl, amino, N-acylamino, nitro, halogen, trifluoromethyl, cyano, and carboxyl (see e.g. Katritzky, Handbook of Heterocyclic Chemistry). If not otherwise indicated, the term “aryl” includes unsubstituted, substituted, and/or heteroatom-containing aromatic substituents.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the terms “heterocyclic” or “heterocycle” refer to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, furyl, pyrimidinyl, imidazolyl, 1,2,4-triazolyl, tetrazolyl, etc., and examples of heteroatom-containing alicyclic groups are pyrrolidino, morpholino, piperazino, piperidino, tetrahydrofuranyl, etc.

“Heteroaryl” refers to an aromatic group of from 1 to 15 carbon atoms, such as from 1 to 10 carbon atoms and 1 to 10 heteroatoms selected from oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

As used herein, the terms “Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 15 ring atoms, including 1 to 4 hetero atoms. These ring atoms are selected from nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycle and heteroaryls include, but are not limited to, 1,3-dioxolane, 1,4-dioxane, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl, —SO₂-heteroaryl, and fused heterocycle.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH2), carbamido (—NH—(CO)—NH2), cyano (—C═N), isocyano (—N+≡C—), cyanato (—O—C≡N), isocyanato (—O—N+≡C—), isothiocyanato (—S—C≡N), azido (—N═N+≡N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO2-alkyl), C5-C20 arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O-M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R^{80′}s, taken together with the nitrogen atom to which they are bonded, form a 5-, 6- or 7-membered heterocycloalkyl which may optionally include from 1 to 4 of the same or different additional heteroatoms selected from O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻ M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺*, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

In certain embodiments, any of the subject compounds disclosed herein may include a deuterium isotopic label.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112. Definitions of other terms and concepts appear throughout the detailed description below.

### DETAILED DESCRIPTION OF THE EMBODIMENTS

Methods of inhibiting the proliferation of a cancer cell, and treating cancer in an individual are provided. Aspects of the subject methods include contacting a cancer cell with an azapodophyllotoxin derivative, where the contacting is effective to inhibit tubulin polymerization and monoglycerol metabolism to inhibit proliferation of cancer in the cell. In certain cases, the cancer cell is a renal cancer cell (RCC) or a lymphoma cell. Aspects of the methods include administering to a subject an effective amount of an azapodophyllotoxin derivative to treat the subject for cancer, where the cancer is selected from renal cancer and lymphoma. In certain cases the administering is effective to inhibit tubulin polymerization and monoglycerol metabolism. Also provided is a method of monitoring tumor regression in an individual. The method of monitoring tumor regression includes assaying, in a sample obtained from the individual during a treatment regime for cancer, changes in tubulin protein levels, wherein a level of tubulin protein that is lower than a pretreatment level of tubulin indicates tumor regression. Also provided are methods of identifying a cancer suppressing compound. Aspects of the methods include contacting a cancer cell with a candidate compound; determining if tubulin protein levels are decreased relative to the cancer cell in the absence of the candidate compound; and determining if monoglycerol levels are increased relative to the cancer cell in the absence of the candidate compound, wherein a decrease in tubulin protein level and an increase in levels of monoglycerols identifies the candidate compound as a cancer suppressing compound. In some cases, the cancer is selected from renal cancer or lymphoma.

### Methods of Inhibiting Tubulin Polymerization and Monoglycerol Metabolism

As summarized above, aspects of the present disclosure include inhibiting proliferation of a cancer cell, by contacting the cell with an azapodophyllotoxin derivative (e.g., as described herein, where the contacting inhibits both tubulin polymerization and monoglycerol metabolism to inhibit proliferation of cancer in the cell. Microtubules are cytoskeletal protein polymers composed of α and β tubulin heterodimers. They are involved in essential cellular processes such as migration, intracellular transport and mitosis (Wade, Molecular biotechnology, 2009; 43(2):177-91; Bates et al., British journal of clinical pharmacology, 2017; 83(2):255-68; Jordan et al., Nature reviews Cancer. 2004; 4(4):253-65; Checchi et al., Trends in pharmacological sciences, 2003; 24(7):361-5). Inhibition of tubulin polymerization leads to disruption of mitotic spindle formation, blocks mitosis and arrests the cell cycle in the G2/M phase, leading to apoptosis. Thus, tubulin is considered as an important target for anticancer drug development. Monoglycerol metabolism has also been implicated in cancer, due to the requirement for energy and building blocks of cancer cells. The inhibition of monoglycerols can impede tumor growth (Beloribi-Djefaflia et al, Oncogenesis. 2016; 5(1): e189).

By inhibiting tubulin polymerization it is meant that the activity of the tubulin protein is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting tubulin polymerization means decreasing the activity of the tubulin protein by a factor of 2 or more, such as 3 or more, 5 or more, 10 or more, 100 or more, or 1000 or more, relative to its normal activity (e.g., relative to a control as measured by any convenient assay).

By inhibiting monoglycerol metabolism it is meant that monoglycerol metabolism is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting monoglycerol metabolism it is meant that monoglycerol metabolism is decreased by a factor of 2 or more, such as 3 or more, 5 or more, 10 or more, 100 or more, or 1000 or more, relative to its normal activity (e.g., relative to a control as measured by any convenient assay).

In some cases, the method is a method of inhibiting tubulin polymerization and monoglycerol metabolism in a sample. The term “sample” as used herein relates to a material or mixture of materials, typically, although not necessarily, in fluid form, containing one or more components of interest.

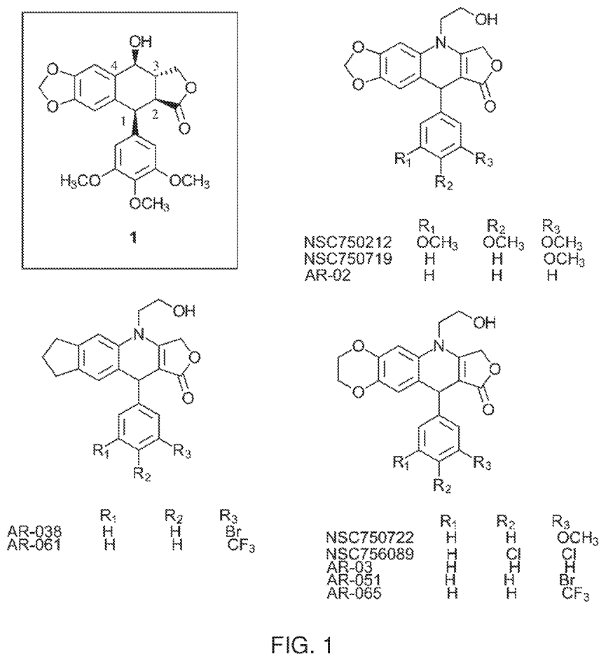

In some embodiments, there is provided a method of inhibiting the proliferation of a cancer cell, the method comprising: contacting the cell with an azapodophyllotoxin derivative; wherein the contacting inhibits tubulin polymerization and monoglycerol metabolism to inhibit proliferation of cancer in the cell. In certain embodiments, the cancer cell is a renal cancer cell (RCC) or a lymphoma cell. In certain embodiments the azapodophyllotoxin derivative is a compound as defined herein. In some embodiments, the azapodophyllotoxin derivative is a compound according to any one of formulas I or VII. In some cases, the azapodophyllotoxin derivative is any one of compounds 1-23. In some cases, azapodophyllotoxin derivative is a structure selected from NSC750212, NSC750719, AR-02, AR-038, AR-061, NSC750722, NSC756089, AR-03, AR-051, and AR-065 (e.g., as shown in FIG. 1).

In some embodiments, the subject compounds have an inhibition profile that reflects activity against additional enzymes. In some embodiments, the subject compounds specifically inhibit tubulin polymerization and monoglycerol metabolism without undesired inhibition of one or more other enzymes.

In some embodiments, the subject compounds inhibit tubulin polymerization, as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting tubulin polymerization. A subject compound (e.g., as described herein) may inhibit at activity of tubulin in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

In some embodiments, the subject compounds inhibit monoglycerol metabolism, as determined by an inhibition assay, e.g., by an assay that determines the level of monoglycerol metabolism either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting monoglycerol metabolism. A subject compound (e.g., as described herein) may inhibit monoglycerol metabolism in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10⁻⁶ M or less (e.g., 1×10⁻⁶ M or less, 1×10⁻⁷ M or less, 1×10⁻⁸ M or less, 1×10⁻⁹ M or less, 1×10⁻¹⁰ M or less, or 1×10⁻¹¹ M or less).

The protocols that may be employed in determining tubulin activity and monoglycerol levels are numerous, and include but are not limited to cell-free assays, e.g., binding assays; assays using purified enzymes, cellular assays in which a cellular phenotype is measured, e.g., gene expression assays; ambient ionization mass spectrometry; and in vivo assays that involve a particular animal (which, in certain embodiments may be an animal model for a condition related to the target pathogen).

In some embodiments, the subject method is an in vitro method that includes contacting a sample with a subject compound that specifically inhibits tubulin polymerization and monoglycerol metabolism. In certain embodiments, the sample is suspected of containing tubulin and monoglycerol and the subject method further comprises evaluating whether the compound inhibits tubulin polymerization and monoglycerol metabolism.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g. biotin), such that any sample that does not bind to the compound may be removed (e.g., by washing).

In some embodiments, the method is a method of reducing cancer cell proliferation, where the method includes contacting the cell with an effective amount of a subject azapodophyllotoxin derivative (e.g., as described herein) to reduce cancer cell proliferation, wherein the cancer cell is a renal cancer cell or a lymphoma cell. The method can be performed in combination with a chemotherapeutic agent (e.g., as described herein). The cancer cells can be in vitro or in vivo. In certain instances, the method includes contacting the cell with a azapodophyllotoxin derivative (e.g., as described herein) and contacting the cell with a chemotherapeutic agent. In certain instances, the method includes contacting the cell with a azapodophyllotoxin derivative in combination with radiation therapy.

### Methods of Treatment

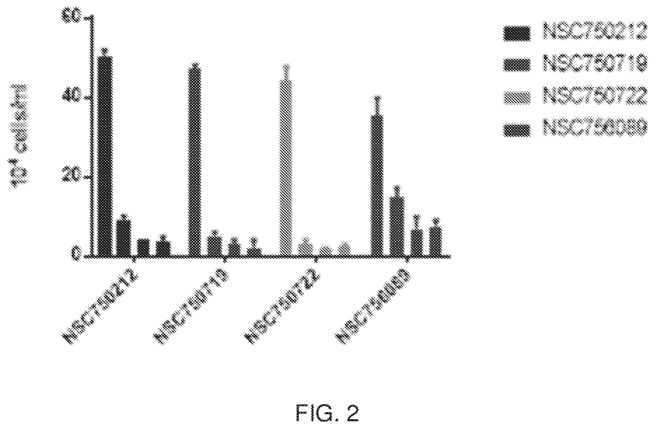

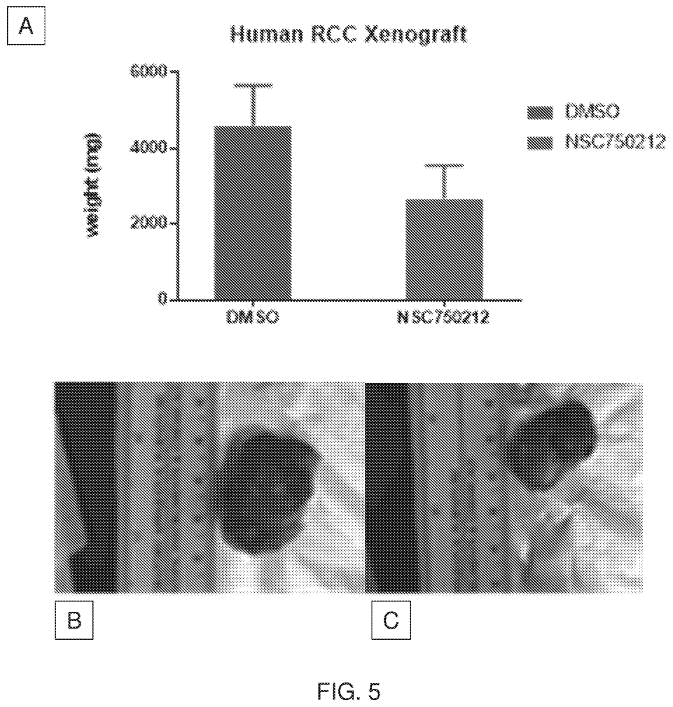

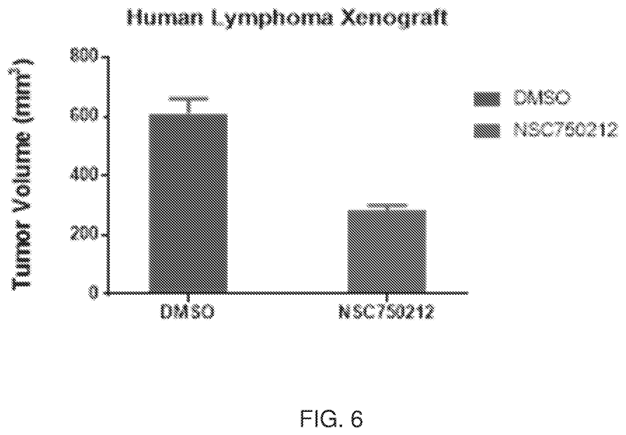

Aspects of the present disclosure include methods for inhibiting tubulin polymerization and monoglycerol metabolism by treatment with a subject compound. The inventors have established that the dual inhibition of tubulin polymerization and monoglycerol metabolism has significant effect on suppressing renal cancer cells (RCC) and lymphoma cell proliferation in vitro (see, e.g., FIG. 2 to FIG. 4), and has a significant effect on tumor regression in renal cancer and lymphoma in vivo (see, e.g., reduction in tumor burden depicted in FIG. 5, panels A-C for exemplary renal cancer xenograft, and FIG. 6 for exemplary lymphoma xenograft).

The results described and demonstrated herein indicate that compounds possessing dual inhibition of tubulin polymerization and monoglycerol metabolism can have a significant impact on suppressing and lymphomas and renal cancer, and thus can overcome the limitations of the therapeutic potential of currently known real cancer and lymphoma treatments. As such, the subject methods provide for potent azapodophyllotoxin derivatives with improved therapeutic potential to treat renal cancer and lymphomas.

As used herein, the terms “renal cancer” or “renal cell carcinoma” refer to cancer that has arisen from the kidney.

The terms “renal cell cancer” or “renal cell carcinoma” (RCC), as used herein, refer to cancer which originates in the lining of the proximal convoluted tubule. More specifically, RCC encompasses several relatively common histologic subtypes: clear cell renal cell carcinoma, papillary (chromophil), chromophobe, collecting duct carcinoma, and medullary carcinoma. Further information about renal cell carcinoma may be found in Y. Thyavihally, et al., Int Semin Surg Oncol 2:18 (2005), the contents of which are incorporated by reference herein. Clear cell renal cell carcinoma (ccRCC) is the most common subtype of RCC. Incidence of ccRCC is increasing, comprising 80% of localized disease and more than 90% of metastatic disease.

As used herein, the term “lymphoma” includes non-Hodgkin lymphomas (NHLs) and Hodgkin Lymphoma (HL). Non-Hodgkin lymphomas are a heterogeneous group of disorders involving malignant monoclonal proliferation of lymphoid cells in lymphoreticular sites, including lymph nodes, bone marrow, the spleen, the liver, and the gastrointestinal tract. Presenting symptoms usually include peripheral lymphadenopathy. Compared with Hodgkin lymphoma, there is a greater likelihood of disseminated disease at the time of diagnosis. However, NHL is not one disease but rather a category of lymphocyte malignancies. These types can be divided into aggressive (fast-growing) and indolent (slow-growing) types, and they can be formed from either B-cells or T-cells. B-cell non-Hodgkin lymphomas include Burkitt lymphoma, chronic lymphocytic leukemia/small lymphocytic lymphoma (CLL/SLL), diffuse large B-cell lymphoma, follicular lymphoma, precursor B-lymphoblastic lymphoma, and mantle cell lymphoma, among others. T-cell non-Hodgkin lymphomas include mycosis fungoides, anaplastic large cell lymphoma, and precursor T-lymphoblastic lymphoma. Lymphomas that occur after bone marrow or stem cell transplantation are usually B-cell non-Hodgkin lymphomas. Prognosis and treatment depend on the stage and type of disease.

Aspects of the methods include administering to a subject in need thereof an effective amount of an azapodophyllotoxin derivative (e.g., as described herein) to treat the subject for cancer, wherein the cancer is selected from renal cancer and lymphoma. Any convenient azapodophyllotoxin derivative can be used in the subject methods of treating renal cancer or lymphoma. In certain cases, the azapodophyllotoxin derivative is a compound as described herein. In certain cases the cancer is a lymphoma. In certain embodiments, the cancer is a renal cancer.

In some embodiments of the methods disclosed herein, the azapodophyllotoxin derivative is a derivative of any one of formulae (I)-(VII). In some cases the azapodophyllotoxin derivative is any one of compounds 1-23. In some cases, azapodophyllotoxin derivative is a structure selected from NSC750212, NSC750719, AR-02, AR-038, AR-061, NSC750722, NSC756089, AR-03, AR-051, and AR-065 (e.g., as shown in FIG. 1).

Aspects of the method include contacting a sample with a subject compound (e.g., as described above) under conditions by which the compound is effective to suppress renal or lymphoma cancer cell proliferation. Any convenient protocol for contacting the compound with the sample may be employed. The particular protocol that is employed may vary, e.g., depending on whether the sample is in vitro or in vivo. For in vitro protocols, contact of the sample with the compound may be achieved using any convenient protocol. In some instances, the sample includes cells that are maintained in a suitable culture medium, and the complex is introduced into the culture medium. For in vivo protocols, any convenient administration protocol may be employed. Depending upon the potency of the compound, the cells of interest, the manner of administration, the number of cells present, various protocols may be employed.

In some embodiments, the subject method is a method of treating a subject for cancer, where the cancer is a renal cancer or a lymphoma. In some embodiments, the subject method includes administering to the subject an effective amount of a subject compound (e.g., as described herein) or a pharmaceutically acceptable salt thereof. The subject compound may be administered as part of a pharmaceutical composition (e.g., as described herein). In certain instances of the method, the compound that is administered is a compound of one of formulae (I)-(VII). In certain instances of the method, the compound that is administered is described by one of the compounds 1-23. In some cases, azapodophyllotoxin derivative is a structure selected from NSC750212, NSC750719, AR-02, AR-038, AR-061, NSC750722, NSC756089, AR-03, AR-051, and AR-065 (e.g., as shown in FIG. 1).

In some embodiments, an “effective amount” is an amount of a subject compound that, when administered to an individual in one or more doses, in monotherapy or in combination therapy, is effective to inhibit tubulin polymerization and monoglycerol metabolism by about 20% (20% inhibition), at least about 30% (30% inhibition), at least about 40% (40% inhibition), at least about 50% (50% inhibition), at least about 60% (60% inhibition), at least about 70% (70% inhibition), at least about 80% (80% inhibition), or at least about 90% (90% inhibition), compared to the tubulin polymerization and monoglycerol metabolism in the individual in the absence of treatment with the compound, or alternatively, compared to the tubulin polymerization and monoglycerol metabolism in the individual before or after treatment with the compound.

In some embodiments, an “effective amount” is an amount of a subject compound that, when administered to an individual in one or more doses, in monotherapy or in combination therapy, is effective to decrease tumor burden in the subject by about 20%, at least about 30%, at least about 40%, at least about 50%, at least about 60%, at least about 70%, at least about 80%, or at least about 90%, compared to tumor burden in the individual in the absence of treatment with the compound, or alternatively, compared to the tumor burden in the subject before or after treatment with the compound. As used herein the term “tumor burden” refers to the total mass of tumor tissue carried by a subject with cancer.

In some embodiments, an “effective amount” of a compound is an amount that, when administered in one or more doses to an individual having renal cancer or lymphoma, is effective to achieve a 1.5-log, a 2-log, a 2.5-log, a 3-log, a 3.5-log, a 4-log, a 4.5-log, or a 5-log reduction in tumor size.

In some embodiments, an effective amount of a compound is an amount that ranges from about 50 ng/kg body weight to about 50 μg/kg body weight (e.g., from about 50 ng/kg body weight to about 40 μg/kg body weight, from about 30 ng/kg body weight to about 20 μg/kg body weight, from about 50 ng/kg body weight to about 10 μg/kg body weight, from about 50 ng/kg body weight to about 1 μg/kg body weight, from about 50 ng/kg body weight to about 800 ng/kg body weight, from about 50 ng/kg body weight to about 700 ng/kg body weight, from about 50 ng/kg body weight to about 600 ng/kg body weight, from about 50 ng/kg body weight to about 500 ng/kg body weight, from about 50 ng/kg body weight to about 400 ng/kg body weight, from about 60 ng/kg body weight to about 400 ng/kg body weight, from about 70 ng/kg body weight to about 300 ng/kg body weight, from about 60 ng/kg body weight to about 100 ng/kg body weight, from about 65 ng/kg body weight to about 85 ng/kg body weight, from about 70 ng/kg body weight to about 90 ng/kg body weight, from about 200 ng/kg body weight to about 900 ng/kg body weight, from about 200 ng/kg body weight to about 800 ng/kg body weight, from about 200 ng/kg body weight to about 700 ng/kg body weight, from about 200 ng/kg body weight to about 600 ng/kg body weight, from about 200 ng/kg body weight to about 500 ng/kg body weight, from about 200 ng/kg body weight to about 400 ng/kg body weight, or from about 200 ng/kg body weight to about 300 ng/kg body weight).

In some embodiments, an effective amount of a compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 pg, from about 1 pg to about 10 pg, from about 10 pg to about 50 pg, from about 50 pg to about 150 pg, from about 150 pg to about 250 pg, from about 250 pg to about 500 pg, from about 500 pg to about 750 pg, from about 750 pg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a compound is administered. In other embodiments, multiple doses are administered. Where multiple doses are administered over a period of time, the compound can be administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Administration of an effective amount of a subject compound to an individual with renal cancer or lymphoma can result in one or more of: 1) a reduction in tumor burden; 2) a reduction in the dose of radiotherapy required to effect tumor shrinkage (e.g. resulting from sensitization to radiotherapy); 3) a reduction in the spread of a cancer from one cell to another cell in an individual; 4) a reduction of morbidity or mortality in clinical outcomes; 5) shortening the total length of treatment when combined with other anti-cancer agents (e.g. resulting from sensitization to other anti-cancer agents); and 6) an improvement in an indicator of disease response (e.g., a reduction in one or more symptoms of the cancer). Any of a variety of methods can be used to determine whether a treatment method is effective. For example, a biological sample obtained from an individual who has been treated with a subject method can be assayed (e.g., as described herein).

Any of the compounds described herein can be utilized in the subject methods of treatment. In some embodiments, the compound specifically inhibits tubulin polymerization and monoglycerol metabolism.

In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys). The subject may be in need of treatment for renal cancer or lymphoma. In some instances, the subject methods include diagnosing cancer, including any one of the cancers described herein. In some embodiments, the compound is administered as a pharmaceutical preparation.

In certain embodiments, the azapodophyllotoxin derivative is a modified compound that includes a label, and the method further includes detecting the label in the subject. The selection of the label depends on the means of detection. Any convenient labeling and detection systems may be used in the subject methods, see e.g., Baker, “The whole picture,” Nature, 463, 2010, p977-980. In certain embodiments, the compound includes a fluorescent label suitable for optical detection. In certain embodiments, the compound includes a radiolabel for detection using positron emission tomography (PET) or single photon emission computed tomography (SPECT). In some cases, the compound includes a paramagnetic label suitable for tomographic detection. The subject compound may be labeled, as described above, although in some methods, the compound is unlabeled and a secondary labeling agent is used for imaging.

### Combination Therapies

The subject compounds can be administered to a subject alone or in combination with an additional, i.e., second, active agent. Combination therapeutic methods where the subject azapodophyllotoxin derivatives may be used in combination with a second active agent or an additional therapy, e.g., radiation therapy. The terms “agent,” “compound,” and “drug” are used interchangeably herein. For example, azapodophyllotoxin derivatives can be administered alone or in conjunction with one or more other drugs, such as drugs employed in the treatment of diseases of interest, including but not limited to, immunomodulatory diseases and conditions and cancer. In some embodiments, the subject method further includes coadministering concomitantly or in sequence a second agent, e.g., a small molecule, a chemotherapeutic, an antibody, an antibody fragment, an antibody-drug conjugate, an aptamer, or a protein. In certain embodiments the second agent is a chemotherapeutic agent. In certain embodiments, the chemotherapeutic agent is a taxane. In some cases, the taxane is paclitaxel. In some embodiments, the method further includes performing radiation therapy on the subject.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug or additional therapy with a pharmaceutical composition of the present disclosure means administration of the compound and second agent or additional therapy at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs or therapies and compounds of the present disclosure.

In some embodiments, the compounds (e.g., a subject compound and the at least one additional compound or therapy) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

In conjunction with any of the subject methods, the azapodophyllotoxin derivatives (e.g., as described herein) (or pharmaceutical compositions comprising such compounds) can be administered in combination with another drug designed to treat a renal cancer or a lymphoma. In certain cases, the cancer is resistant to the drug. In certain cases, the azapodophyllotoxin derivative can be administered prior to, at the same time as, or after the administration of the other drug.

For the treatment of cancer, the azapodophyllotoxin derivatives can be administered in combination with a chemotherapeutic agent selected from alkylating agents, nitrosoureas, antimetabolites, antitumor antibiotics, plant (vinca) alkaloids, steroid hormones, taxanes, nucleoside analogs, steroids, anthracyclines, thyroid hormone replacement drugs, thymidylate-targeted drugs, Chimeric Antigen Receptor/T cell therapies, Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCAR1 (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine, or a genetically modified tyrosinase such as Oncept®) and other cell therapies.

Specific chemotherapeutic agents of interest include, but are not limited to, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, and ABT-199. Peptidic compounds can also be used. Cancer chemotherapeutic agents of interest include, but are not limited to, taxane and active analogs and derivatives thereof. As used herein, the term “taxane” refers to compounds that have the basic taxane skeleton as a common structure feature. In certain embodiments, the taxane is paclitaxel. Paclitaxel is a highly derivatized diterpenoid (Wani, et al. (1971) J. Am. Chem. Soc. 93:2325-2327) which has been obtained from the harvested and dried bark of Taxus brevifolia (Pacific Yew) and Taxomyces andreanae, an endophytic fungus of the Pacific Yew (Stierle, et al. (1993) Science 60:214-216). Also included in the term “taxanes” are paclitaxel analogues, formulations, and derivatives, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel. As such, the term taxane refers to not only the common chemically available form of paclitaxel, but analogs (e.g., taxotere, as noted above) and paclitaxel conjugates (e.g., paclitaxel-PEG, paclitaxel-dextran, or paclitaxel-xylose). Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

In some embodiments, the azapodophyllotoxin compounds can be administered in combination with a chemotherapeutic agent to treat a renal cancer or lymphoma. In certain cases, the chemotherapeutic agent is a taxane. In some cases, the chemotherapeutic agent is paclitaxel.

Any convenient cancer vaccine therapies and agents can be used in combination with the subject azapodophyllotoxin derivatives, compositions and methods. For treatment of cancer, e.g., renal cancer, or lymphoma, the azapodophyllotoxin derivative can be administered in combination with a vaccination therapy, e.g., a dendritic cell (DC) vaccination agent that promotes Th1/Th17 immunity. Th17 cell infiltration correlates with markedly prolonged overall survival among ovarian cancer patients. In some cases, the azapodophyllotoxin derivative finds use as adjuvant treatment in combination with Th17-inducing vaccination.

In certain instances, the combination provides an enhanced effect relative to either component alone; in some cases, the combination provides a supra-additive or synergistic effect relative to the combined or additive effects of the components. A variety of combinations of the subject compounds and the chemotherapeutic agent may be employed, used either sequentially or simultaneously. For multiple dosages, the two agents may directly alternate, or two or more doses of one agent may be alternated with a single dose of the other agent, for example. Simultaneous administration of both agents may also be alternated or otherwise interspersed with dosages of the individual agents. In some cases, the time between dosages may be for a period from about 1-6 hours, to about 6-12 hours, to about 12-24 hours, to about 1-2 days, to about 1-2 week or longer following the initiation of treatment.

### Azapodophyllotoxin Derivatives

As summarized above, the subject methods include azapodophyllotoxin derivatives. Exemplary derivatives including azapodophyllotoxin core structures are set forth in the following structures 1-23 and formulae I-VII. Exemplary derivatives including azapodophyllotoxin core structures are also set forth in FIG. 1, e.g., a compound selected from NSC750212, NSC750719, AR-02, AR-038, AR-061, NSC750722, NSC756089, AR-03, AR-051, and AR-065.

Podophyllotoxin (FIG. 1, (1)), a lignan obtained from plants of the Podophyllum genus, is an important ligand with remarkable microtubule assembly inhibitory activity. However, its therapeutic use has been restricted due to its high toxicity (Imbert et al., Biochimie. 1998; 80(3):207-22). Extensive efforts to reduce its toxic effects led to development of its semisynthetic derivatives etoposide and teniposide, which are currently used in combination cancer chemotherapy, but have different mechanism of action then podophyllotoxin (Han et al., Chemistry & biodiversity. 2018; 15(11):e1800289; and Cheng et al., Molecules. 2015; 20(7):12266-79). Reports on completely synthetic analogues of podophyllotoxins are rather limited due to its highly complex structure. As disclosed herein, the inventors have developed azapodophyllotoxin derivatives (FIG. 1) which exhibited remarkable anticancer activities. These compounds are structurally simpler than podophyllotoxin, containing only one stereocenter, and were synthesized in a single step in good yields from commercially available starting materials.

Disclosed herein are the in vitro cancer suppression, and in vivo efficacy of exemplary azapodophyllotoxin derivatives in both renal cancer cells and lymphoma cells. Also presented herein is a mechanistic investigation demonstrating that exemplary azapodophyllotoxin derivatives can exhibit a dual mode of action as inhibitors of tubulin polymerization and monoglycerol metabolism.

In some embodiments there is provided a azapodophyllotoxin derivative of formula (I):

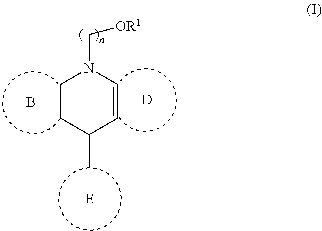

wherein:

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

Ring B and Ring E are each independently selected from a C₅₋₆ membered carbocycle, a substituted C₅₋₆ membered carbocycle a C₅₋₆ membered heteroaryl, a substituted C₅₋₆ membered heteroaryl, a C₅₋₆ membered heterocycle containing up to two atoms selected from N, O or S and a substituted C₅₋₆ membered heterocycle containing up to two atoms selected from N, O or S (e.g. pyrrole, imidazole, pyrazole, furan, oxazole, isoxazole, thiophene, thiazole, isothiazole, pyridine, pyrimidine, 2-H-pyran, 2-H-thiopyran);

Ring D is selected from a C₅₋₆ carbocycle, a C₅₋₆ heterocycle containing up to two atoms selected from N, O or S, a substituted C₅₋₆ carbocycle, and a substituted C₅₋₆ heterocycle containing up to two atoms selected from N, O or S (e.g. 2-furanone, 1,3-dioxolane, cyclopentane, cyclopentene, 1,4-dioxane, cyclohexane, cyclohexene, cyclohexanone); and

n is an integer from 1 to 6,

or a pro-drug, a pharmaceutically acceptable salt or a solvate thereof.

In some embodiments of formula (I), R¹ is H. In other embodiments, R¹ is a substituent other than H, such as alkyl, alkoxy, carbocycle, heterocycle, heteroaryl or a protecting group, each of which may be optionally further substituted with one or more substituents.

In certain embodiments of formula I the D ring is selected from:

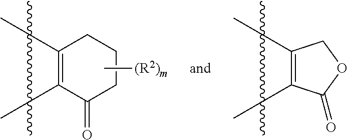

wherein:

each R² are independently selected from alkyl, substituted alkyl, acyl, substituted acyl, alkoxy, substituted alkoxy, carbocycle, substituted carbocycle, heterocycle, substituted heterocycle, heteroaryl, substituted heteroaryl, F, CF₃, CN, NO₂ and methoxy; and

m is an integer from 0 to 6. Thus, the compound of formula (I) may be a compound of formula (IA) or (IB):

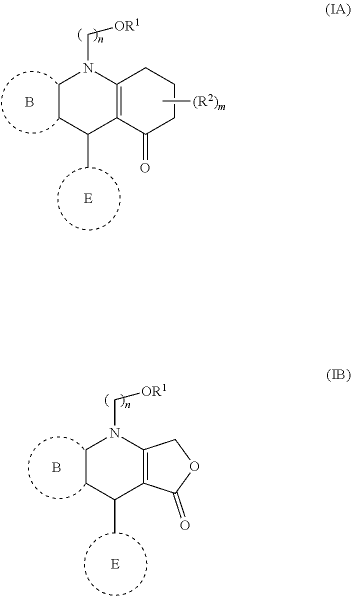

wherein the Ring B and Ring E are each independently any of the groups as defined above for a compound of formula (I).

In certain embodiments of a azapodophyllotoxin derivative of any of formulas (I)-(IB), the B ring or E ring are each independently selected from aryl, substituted aryl, pyrrole, substituted pyrrole, imidazole, substituted imidazole, pyrazole, substituted pyrazole, furan, substituted furan, oxazole, substituted oxazole, isoxazole, substituted isoxazole, thiophene, substituted thiophene, thiazole, substituted thiazole, isothiazole, substituted isothiazole, pyridine, substituted pyridine, pyrimidine, substituted pyrimidine, 2-H-pyran, substituted 2-H-pyran, 2-H-thiopyran and substituted 2-H-thiopyran.

In some cases of a azapodophyllotoxin derivative of any of formulas (I)-(IB), the B ring is of the formula (B1)

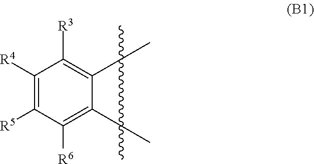

wherein:

R³, R⁴, R⁵ and R⁶ are each independently selected from H, OH, methoxy, halogen, CF₃, CN and NO₂;

or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S (e.g. 2-furanone, 1,3-dioxolane, cyclopentane, cyclopentene, 1,4-dioxane, cyclohexane, cyclohexene, cyclohexanone). Thus, the compound of formula (I) may be a compound of formulae (IC), (ID) or (IE):

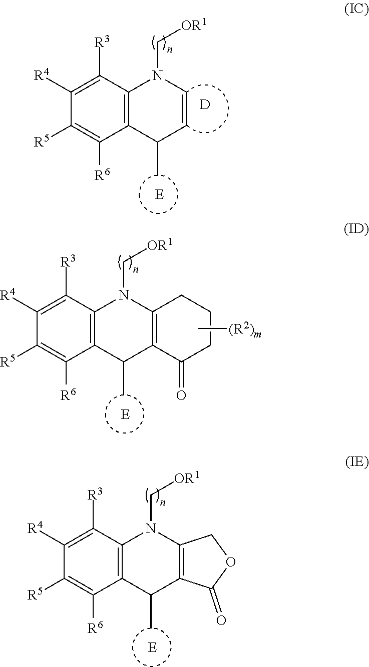

wherein the Ring D and Ring E are each independently any of the groups as defined above for a compound of formula (I).

In some cases of a azapodophyllotoxin derivative of any of formulas (I)-(IE), the E ring is of the formula (E1):

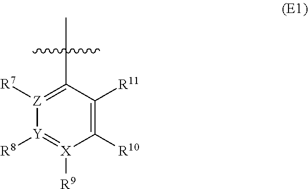

wherein:

X, Y and Z are each independently selected from C or N; and

R⁷, R⁸, R⁹, R¹⁰ and R¹¹ are each independently selected from H, F, CF₃, CN, NO₂, Cl, Br, OH and alkyl, and alkoxy (e.g., methoxy);

or R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S. (e.g. 2-furanone, 1,3-dioxolane, cyclopentane, cyclopentene, 1,4-dioxane, cyclohexane, cyclohexene, cyclohexanone, pyrrole, imidazole, pyrazole, pyrrolidine, imidazoline, tetrahydrofurane, furane, oxazole, isoxazole, thiolane, isoxazole, thiophene, thiazole, isothiazole, pyridine, pyrimidine, 1,4-piperazine, piperidine, morpholine, 1,4-dithiane). Thus, the compound of formula (I) may be a compound of any one of formulae (IF) to (IL):

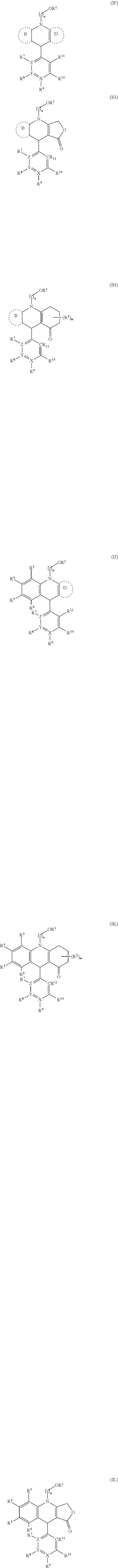

wherein the Ring B, Ring D and Ring E are each independently any of the groups as defined above for a compound of formula (I).

In certain embodiments of any one of formulae (I)-(IL), n is an integer of 6 or less, such as 5, 4, 3, 2 or 1. In certain cases of any one of formulae (I)-(IL), n is 2.

In some cases of a azapodophyllotoxin derivative of any of formulas (I)-(IB), the B ring is of the formula (B2) or (B3):

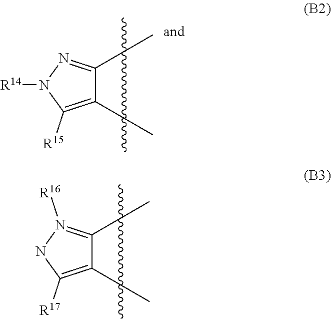

wherein:

R¹⁴, R¹⁵, R¹⁶ and R¹⁷ are each independently selected from H, alkyl, aryl, substituted aryl. Thus, the compound of formula (I) may be a compound of any one of formulae (IM) to (IR):

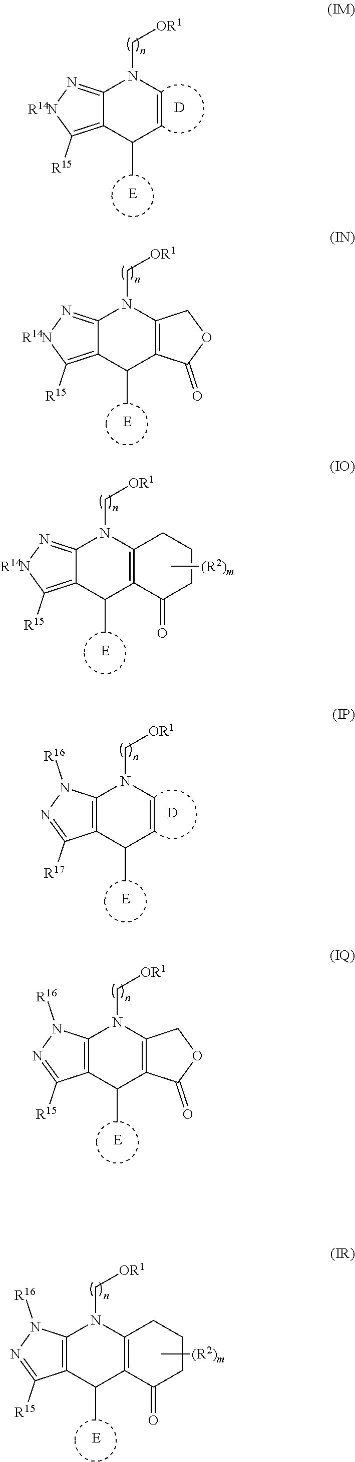

wherein the Ring D and Ring E are each independently any of the groups as defined above for a compound of formula (I).

In some cases, the azapodophyllotoxin derivative of formula (I) may be a compound of any one of formulae (IS) to (IV):

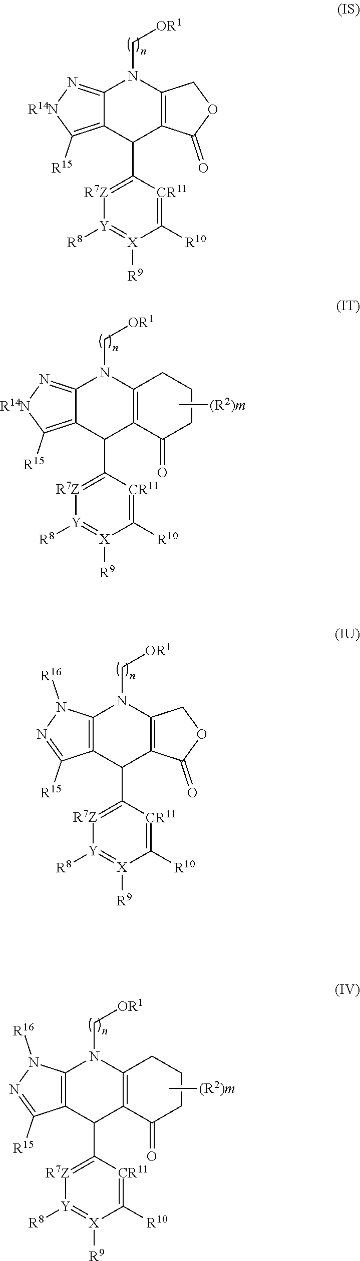

In certain embodiments of any one of formulae (IM)-(IV), n is an integer of 6 or less, such as 5, 4, 3, 2 or 1. In certain cases of any one of formulae (IM)-(IV), n is 2.

In some embodiments, the azapodophyllotoxin derivative of formula (I), is of the formula (II):

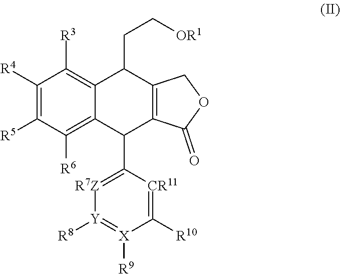

wherein:

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group; R³, R⁴, R⁵ and R⁶ are independently selected from H, OH, methoxy, alkyl, halogen, CF₃, CN and NO₂;

or any of R¹ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S (e.g. 2-furanone, 1,3-dioxolane, cyclopentane, cyclopentene, 1,4-dioxane, cyclohexane, cyclohexene, cyclohexanone);

X, Y and Z are each independently selected from C or N;

R¹⁰ is selected from H, F, CF₃, CN, NO₂, OH, methoxy and alkyl;

R⁷, R⁸, R⁹ and R¹¹ are each independently selected from H, F, CF₃, CN, NO₂, methoxy, Cl, Br, OH and alkyl;

or R⁷ and R⁸, R⁸ and R⁹, R⁹ and R¹⁰, R¹⁰ and R¹¹ together with the carbons to which they are attached form a C₅₋₆ carbocycle, or C₅₋₆ heterocycle containing up to two atoms selected from N, O or S a substituted C₅₋₆ carbocycle, or a substituted C₅₋₆ membered heterocycle containing up to two atoms selected from N, O or S,

or a pro-drug, a pharmaceutically acceptable salt or a solvate thereof.

In some embodiments of formula (II), at least one of R⁷-R¹¹ is F. In some cases of formula (II), at least one of R⁷-R¹¹ is CF₃. In other cases, of formula (II), at least one of R⁷-R¹¹ is NO₂. In some embodiments of formula (II), at least one of R⁷-R¹¹ is CN. In certain cases of formula (II), at least one of R⁷-R¹¹ is a group other than H. In certain cases of formula (II), at least two of R⁷-R¹¹ are groups other than hydrogen. In certain instances, R⁸, R⁹ and R¹⁰ are groups other than hydrogen and R⁷ and R¹¹ are both hydrogen.

In certain instances, the azapodophyllotoxin is a structure selected from NSC750212, NSC750719, AR-02, AR-038, AR-061, NSC750722, NSC756089, AR-03, AR-051, and AR-065 (e.g., as shown in FIG. 1). In certain cases, the azapodophyllotoxin derivative is NSC750212. In certain cases, the azapodophyllotoxin derivative is NSC750719. In certain cases, the azapodophyllotoxin derivative is AR-02. In certain cases, the azapodophyllotoxin derivative is AR-038. In certain cases, the azapodophyllotoxin derivative is AR-061. In certain cases, the azapodophyllotoxin derivative is NSC750722. In certain cases, the azapodophyllotoxin derivative is NSC756089. In certain cases, the azapodophyllotoxin derivative is AR-03. In certain cases, the azapodophyllotoxin derivative is AR-051. In certain cases, the azapodophyllotoxin derivative is AR-065.

In certain instances of the azapodophyllotoxin derivative of formula (II), all of X, Y and Z are carbon atoms. In other cases of the azapodophyllotoxin derivatives of formula (II), at least one of X, Y or Z is a nitrogen atom. In other cases of the azapodophyllotoxin derivatives of formula (II), X is a nitrogen atom and Y and Z are both carbon atoms. In other cases of the azapodophyllotoxin derivatives of formula (II), Y is a nitrogen atom and X and Z are both carbon atoms. In other cases of the azapodophyllotoxin derivatives of formula (II), Z is a nitrogen atom and X and Y are both carbon atoms.

In certain cases, the azapodophyllotoxin derivative of formula (I), is of the formula (III):

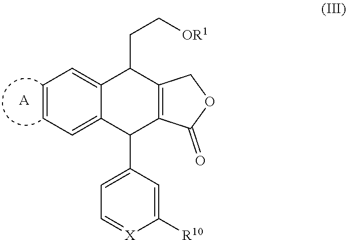

wherein:

Ring A is selected from a C₅₋₆ carbocycle, a C₅₋₆ heterocycle containing up to two atoms selected from N, O or S, a substituted C₅₋₆ carbocycle, and a substituted C₅₋₆ heterocycle containing up to two atoms selected from N, O or S;

X is C or N;

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

R¹⁰ is selected from F, CF₃, CN, NO₂, OH, alkyl and methoxy,

or a pro-drug, a pharmaceutically acceptable salt or a solvate thereof.

In certain instances of the azapodophyllotoxin derivative of formula (III), the A Ring is selected from 1,3-dioxolane, cyclopentane, cyclopentene, 1,4-dioxane, cyclohexane, cyclohexene. In some cases, the A ring is dioxolane. In some cases, the A ring is cyclopentane. In some cases, the A ring is cyclopentane. In some cases, the A ring is 1,4-dioxane. In some cases, the A ring is cyclohexane. In some other cases, the A ring is cyclohexene.

In certain instances of the azapodophyllotoxin derivative of formula (III), R¹⁰ is F. In other cases of formula (III), R¹⁰ is CF₃. In other cases of formula (III), R¹⁰ is Br. In other cases of formula (III), R¹⁰ is Cl. In other cases of formula (III), R¹⁰ is CN. In yet other cases of formula (III), R¹⁰ is NO₂. In some cases R¹⁰ is OH. In some cases, R¹⁰ is alkyl. In some cases, R¹⁰ is methoxy.

In certain instances of the azapodophyllotoxin derivative of formula (III), X is a carbon atom. In other cases of the azapodophyllotoxin derivative of formula (III), X is a nitrogen atom. In other cases of the azapodophyllotoxin derivative of formula (III), X is an oxygen atom. In other cases of the azapodophyllotoxin derivative of formula (III), X is a sulfur atom.

In some embodiments the azapodophyllotoxin derivative of formula (III) is a structure selected from any of compounds (1)-(11):

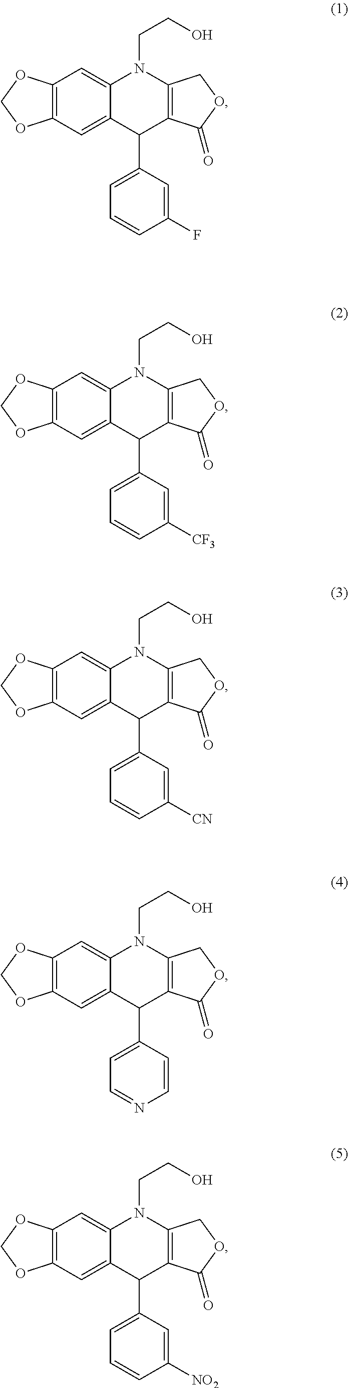 [SMILES: O=C1OCC2=C1C(c1cccc(F)c1)c1cc3c(cc1N2CCO)OCO3 ; O=C1OCC2=C1C(c1cccc(C(F)(F)F)c1)c1cc3c(cc1N2CCO)OCO3 ; N#Cc1cccc(C2C3=C(COC3=O)N(CCO)c3cc4c(cc32)OCO4)c1 ; O=C1OCC2=C1C(c1ccncc1)c1cc3c(cc1N2CCO)OCO3 ; O=C1OCC2=C1C(c1cccc([N+](=O)[O-])c1)c1cc3c(cc1N2CCO)OCO3]

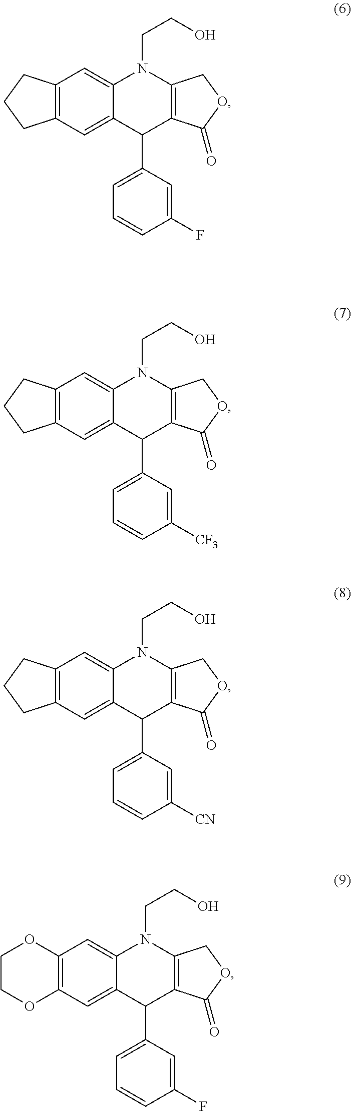 [SMILES: O=C1OCC2=C1C(c1cccc(F)c1)c1cc3c(cc1N2CCO)CCC3 ; O=C1OCC2=C1C(c1cccc(C(F)(F)F)c1)c1cc3c(cc1N2CCO)CCC3 ; N#Cc1cccc(C2C3=C(COC3=O)N(CCO)c3cc4c(cc32)CCC4)c1 ; O=C1OCC2=C1C(c1cccc(F)c1)c1cc3c(cc1N2CCO)OCCO3]

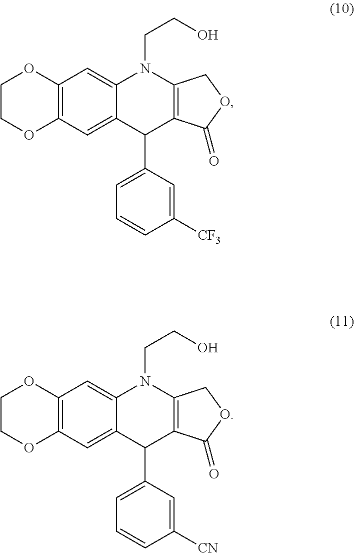 [SMILES: O=C1OCC2=C1C(c1cccc(C(F)(F)F)c1)c1cc3c(cc1N2CCO)OCCO3 ; N#Cc1cccc(C2C3=C(COC3=O)N(CCO)c3cc4c(cc32)OCCO4)c1]

In some embodiments the azapodophyllotoxin derivative of formula (II) is a structure selected from any of compounds (12)-(16):

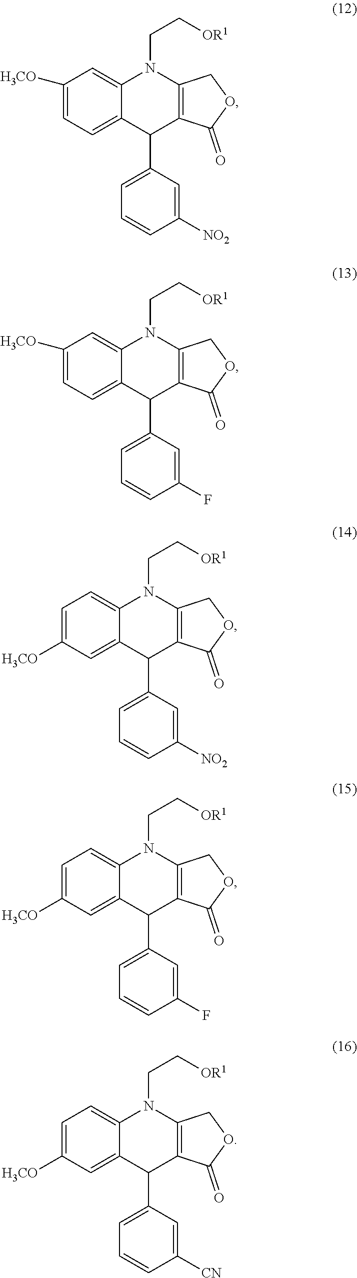

In some embodiments, the azapodophyllotoxin derivative of formula (I), is of the formula (IV):

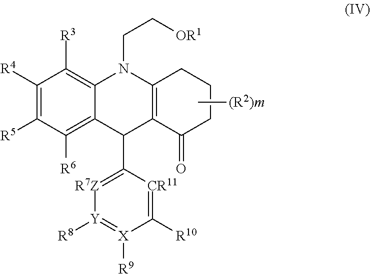

wherein:

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

R² is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

R³, R⁴, R⁵ and R⁶ are independently selected from H, OH, methoxy, alkyl, halogen, CF₃, CN and NO₂;

or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S (e.g. 2-furanone, 1,3-dioxolane, cyclopentane, cyclopentene, 1,4-dioxane, cyclohexane, cyclohexene, cyclohexanone);

X, Y and Z are each independently selected from C or N;

R⁷, R⁸, R⁹, R¹⁰ and R¹¹ are each independently selected from H, F, CF₃, CN, NO₂, methoxy, Cl, Br, OH and alkyl;

or R⁷ and R⁸, R⁸ and R⁹, R⁹ and R¹⁰, R¹⁰ and R¹¹ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S; and

m is an integer from 0 to 6,

or a pro-drug, a pharmaceutically acceptable salt or a solvate thereof.

In some embodiments of formula (IV), at least one of R⁷-R¹¹ is F. In some embodiments of formula (IV), at least one of R⁷-R¹¹ is methoxy. In some cases of formula (IV), at least one of R⁷-R¹¹ is CF₃. In other cases, of formula (IV), at least one of R⁷-R¹¹ is NO₂. In some embodiments of formula (IV), at least one of R⁷-R¹¹ is CN. In certain cases of formula (IV), at least one of R⁷-R¹¹ is a group other than hydrogen. In certain cases of formula (IV), all of R⁷-R¹¹ are hydrogen. In certain cases of formula (IV), at least two of R⁷-R¹¹ are groups other than hydrogen. In certain instances of formula (IV), R⁸, R⁹ and R¹⁰ are groups other than hydrogen and R⁷ and R¹¹ are both hydrogen. In certain instances of formula (IV), R⁸, R⁹ and R¹⁰ are methoxy groups and R⁷ and R¹¹ are both hydrogen.

In certain instances of the azapodophyllotoxin derivative of formula (IV), all of X, Y and Z are carbon atoms. In other cases of the azapodophyllotoxin derivative of formula (IV), at least one of X, Y or Z is a nitrogen atom. In other cases of the azapodophyllotoxin derivative of formula (IV), X is a nitrogen atom and Y and Z are both carbon atoms. In other cases of the azapodophyllotoxin derivative of formula (IV), Y is a nitrogen atom and X and Z are both carbon atoms. In other cases of the azapodophyllotoxin derivative of formula (IV), Z is a nitrogen atom and X and Y are both carbon atoms.

In certain cases of formula (IV), m is 0. In some cases of formula (IV), m is from 1-6 and each R² is independently selected from an alkyl, a substituted alkyl or a combination thereof. In some embodiments of formula (IV), m is 2 and each R² is a C₁₋₆ alkyl. In certain cases of formula (IV), m is 2 and each R² is methyl. In other case m is 1 and R² is methyl. In other cases, m is 2 and R² is ethyl. In other case m is 1 and R² is ethyl. In other cases, m is 2 and R² is ethyl. In other case m is 1 and R² is ethyl. In other cases, m is 2 and R² is propyl. In other case m is 1 and R² is propyl. In other cases, m is 2 and R² is butyl. In other case m is 1 and R² is butyl. In other cases, m is 2 and R² is pentyl. In other case m is 1 and R² is pentyl. In yet other cases, m is 2 and R² is hexyl. In other case m is 1 and R² is hexyl.

In some embodiments the azapodophyllotoxin derivative of the formula (IV), is of the formula (V):

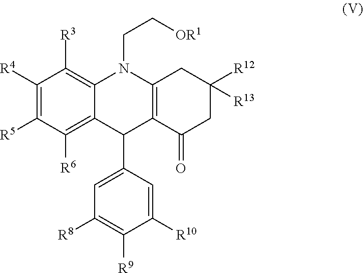

wherein:

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

R³, R⁴, R⁵ and R⁶ are independently selected from H, OH, methoxy, alkyl, halogen, CF₃, CN and NO₂;

or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S (e.g. 2-furanone, 1,3-dioxolane, cyclopentane, cyclopentene, 1,4-dioxane, cyclohexane, cyclohexene, cyclohexanone);

R¹² and R¹³ are each independently selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl, F, CF₃, CN, NO₂ and methoxy;

R⁸, R⁹ and R¹⁰ are each independently selected from H, F, CF₃, CN, NO₂, methoxy, Cl, Br, OH and alkyl;

or any of R⁸ and R⁹ or R⁹ and R¹⁰ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S,

or a pro-drug, a pharmaceutically acceptable salt or a solvate thereof.

In some cases of formulae (IV) or (V), R⁴ is methoxy and each of R³, R⁵ and R⁶ are H. In other cases of formulae (IV) or (V), each of R³, R⁴ and R⁶ are H and R⁵ is methoxy. In other cases of formulae (IV) or (V), R⁴ and R⁵ together with the carbons to which they are attached form a group selected from 1,3-dioxolane, cyclopentane, cyclopentene, 1,4-dioxane, cyclohexane, cyclohexene; and each of R³ and R⁶ are H. In yet other cases of formulae (IV) or (V), R⁴ and R⁵ together with the carbons to which they are attached form 1,3-dioxolane; and each of R³ and R⁶ are H. In yet other cases of formulae (IV) or (V), R⁴ and R⁵ together with the carbons to which they are attached form cyclopentane; and each of R³ and R⁶ are H.

In certain embodiments of formula (V), R¹⁰ is F and R⁸ and R⁹ are both hydrogen. In other cases of formula (V), R⁸, R⁹ and R¹⁰ are each hydrogen. In some other instances, R⁸, R⁹ and R¹⁰ are each methoxy.

In certain cases of formula (V), R¹² and R¹³ are both hydrogen. In some cases of formula (V), R¹² and R¹³ are both independently selected from an alkyl, a substituted alkyl or a combination thereof. In some embodiments of formula (V), R¹² and R¹³ are both C₁₋₆ alkyl. In some embodiments of formula (V), R¹² and R¹³ are both methyl.

In some embodiments the azapodophyllotoxin derivative of formula (V) is a structure selected from any of compounds (17)-(23):

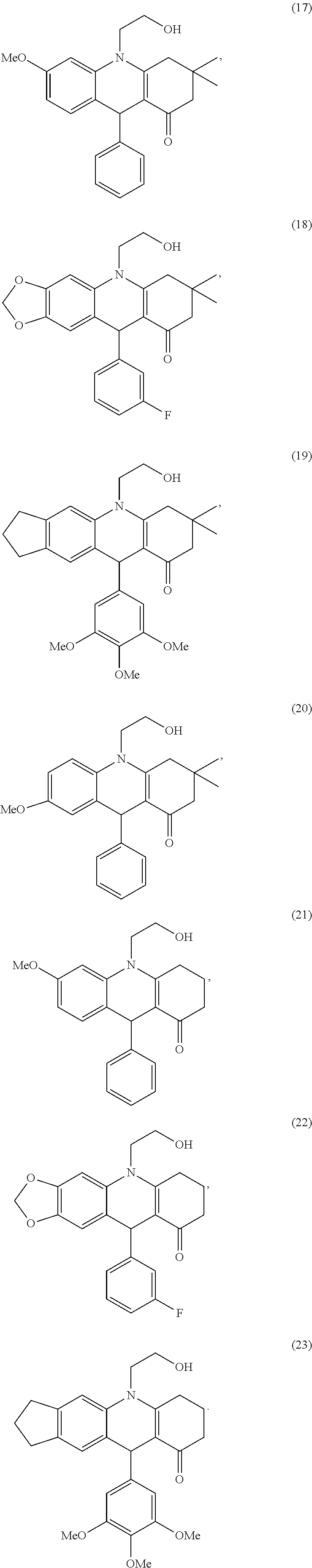

In some embodiments, the azapodophyllotoxin derivative of formula (I), is of the formula (VI):

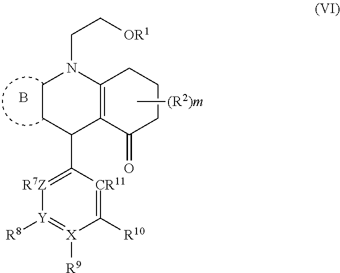

wherein:

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

R² is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

X, Y and Z are each independently selected from C or N;

R⁷, R⁸, R⁹, R¹⁰ and R¹¹ are each independently selected from H, F, CF₃, CN, NO₂, methoxy, Cl, Br, OH and alkyl;

or any of R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S;

Ring B is selected from the formulae (B2) and (B3):

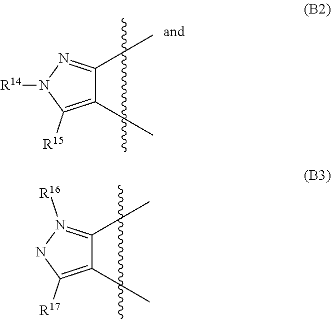

wherein R¹⁴, R¹⁵, R¹⁶ and R¹⁷ are each independently selected from H, alkyl, aryl, substituted aryl;

and

m is an integer from 0 to 6,

or a pro-drug, a pharmaceutically acceptable salt or a solvate thereof.

In certain embodiments of formula (VI), Ring B is of the formula (B2). In other embodiments of formula (VI), Ring B is of the formula (B3). Accordingly, in certain cases the compound of formula (VI) is of the formulae (VIA) or (VIB):

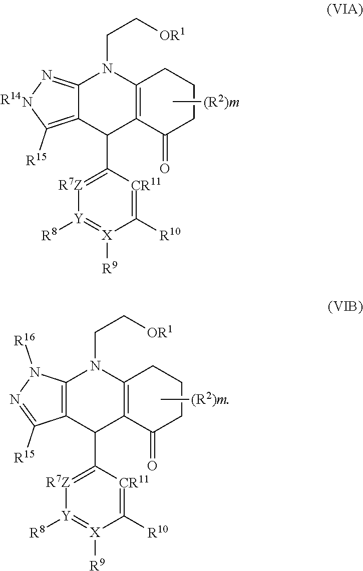

In certain embodiments the compound of formula (VI) is of the formulae (VIC) or (VID):

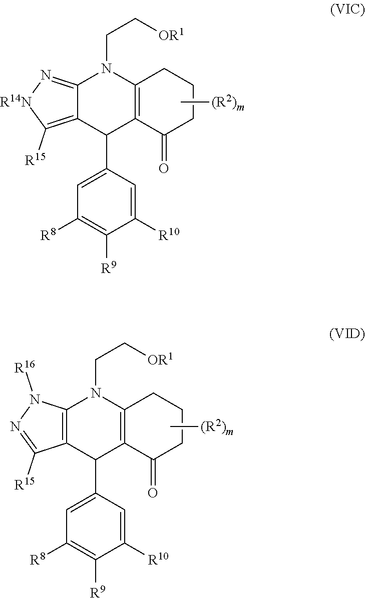

wherein each of the groups R¹, R², R⁸, R⁹, R¹⁰, R¹⁴, R¹⁵ and R¹⁶ are as defined herein.

In some embodiments, the azapodophyllotoxin derivative of formula (I), is of the formula (VII):

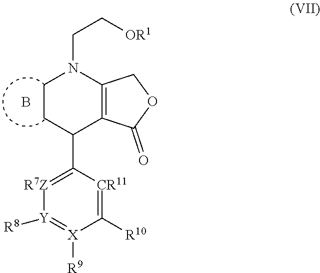

wherein:

R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;

X, Y and Z are each independently selected from C or N;

R⁷, R⁸, R⁹, R¹⁰ and R¹¹ are each independently selected from H, F, CF₃, CN, NO₂, methoxy, Cl, Br, OH and alkyl;

or any of R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S; and

Ring B is selected from the formulae (B2) and (B3):

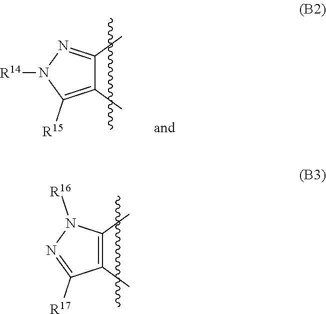

wherein R¹⁴, R¹⁵, R¹⁶ and R¹⁷ are each independently selected from H, alkyl, aryl, and substituted aryl;

or a pro-drug, a pharmaceutically acceptable salt or a solvate thereof.

In certain embodiments of formula (VII), Ring B is of the formula (B2). In other embodiments of formula (VII), Ring B is of the formula (B3). Accordingly, in certain cases the compound of formula (VII) is of the formulae (VIIA) or (VIIB):

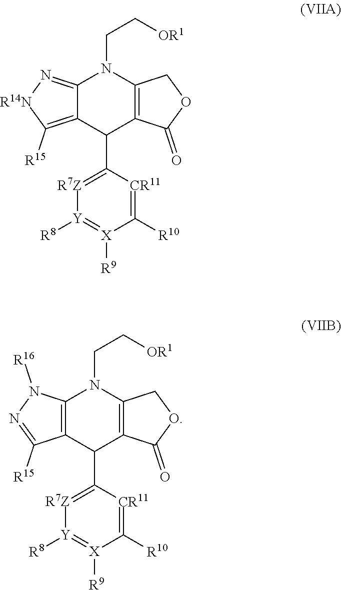

In certain embodiments the compound of formula (VII) is of the formulae (VIIC) or (VIID):

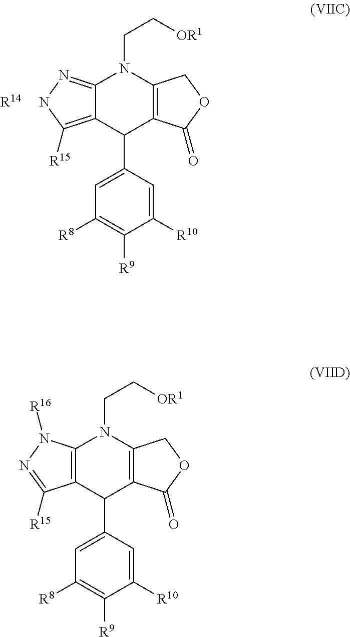

wherein each of the groups R¹, R⁸, R⁹, R¹⁰, R¹⁴, R¹⁵ and R¹⁶ are as defined herein.

In certain embodiments, the compound is described by the structure of one of the compounds of 1-23, or a compound of FIG. 1 selected from NSC750212, NSC750719, AR-02, AR-038, AR-061, NSC750722, NSC756089, AR-03, AR-051, and AR-065. It is understood that any of the subject compounds may be present in a salt form. In some cases, the salt form of the compound is a pharmaceutically acceptable salt.

In certain embodiments, the compound described by any one of compounds 1-23, or a compound of FIG. 1 selected from NSC750212, NSC750719, AR-02, AR-038, AR-061, NSC750722, NSC756089, AR-03, AR-051, and AR-065 is an enantiomerically pure compound.

Aspects of the present disclosure include azapodophyllotoxin derivatives (e.g., as described herein), salts thereof (e.g., pharmaceutically acceptable salts), and/or solvate, hydrate and/or prodrug forms thereof. It is understood that, in any compound described herein having one or more chiral centers, if an absolute stereochemistry is not expressly indicated, then each center may independently be of R-configuration or S-configuration or a mixture thereof. More specifically, where compounds described herein contain one or more chiral centers and/or double-bond isomers (i.e., geometric isomers), enantiomers or diastereomers, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure. It will be appreciated that all permutations of salts, solvates, hydrates, prodrugs and stereoisomers are meant to be encompassed by the present disclosure.

In some embodiments, the subject azapodophyllotoxin derivatives, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)). In some cases, the promoiety is attached to a hydroxy group of the subject compounds.

In some embodiments, any of the subject compound disclosed herein is in a prodrug form, wherein R1 includes, but is not limited to, a group selected from phosphate, —O—CR₂OP(O)O₂M, where M is H, or an ionic salt, and R is H, or lower alkyl; an acidic moiety (e.g., CO₂H, PO₃H₂ or SO₃H etc.) tethered to an ester attached to the parent molecule; an amino moiety, or ammonium moiety tethered to an ester attached to the parent molecule; an alcohol or polyol tethered to an ester attached to the parent molecule; a PEG ester, a sugar ester, an amino acid ester; 4-nitrophenyl methylene carbonates; a gamma amino butyric acid ester, a nitrate ester.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some embodiments, the subject compounds are provided by oral dosing and absorbed into the bloodstream. In some embodiments, the oral bioavailability of the subject compounds is 30% or more. Modifications may be made to the subject compounds or their formulations using any convenient methods to increase absorption across the gut lumen or their bioavailability.

In some embodiments, the subject compounds are metabolically stable (e.g., remain substantially intact in vivo during the half-life of the compound). In certain embodiments, the compounds have a half-life (e.g., an in vivo half-life) of 5 minutes or more, such as 10 minutes or more, 12 minutes or more, 15 minutes or more, 20 minutes or more, 30 minutes or more, 60 minutes or more, 2 hours or more, 6 hours or more, 12 hours or more, 24 hours or more, or even more.

### Monitoring Methods

Aspects of the present disclosure includes methods for monitoring tumor regression in the individual. The monitoring methods include assaying a sample obtained from an individual during a treatment regime for cancer for changes in tubulin protein levels, where a level of tubulin protein that is lower than a pre-treatment level of tubulin indicates tumor regression.

As both a way of quantifiably comparing the effects of an exemplary azapodophyllotoxin derivative at the protein level, and a way of monitoring changes in the proteomic landscape during treatment, a nano-fluidic proteomic immunoassay (NIA) can be used (Fan et al., Expert opinion on investigational drugs. 2013; 22(11):1495-509; and Fan et al., Nature medicine. 2009; 15(5):566-71). This therapeutic monitoring technique is a highly sensitive method for measuring protein expression in clinical samples. Herein, NIA is further developed to be able to analyze fine needle aspirates (FNA's) and core biopsies of transplanted xenografts to monitor changes in tubulin protein levels throughout the course of treatment of mice with an exemplary azapodophyllotoxin derivative to determine whether there is a significant quantifiable difference between the treatment group in comparison with the control.

To monitor the effects of exemplary azapodophyllotoxin derivatives on cancer metabolism, ambient ionization mass spectrometry is utilized. Ambient ionization mass spectrometry is a collective term describing all mass spectrometric ionization methods that are capable of ionizing the constituents of natural samples under ambient conditions. In this regard, ambient mass spectrometry (MS) methods are well-suited for studying tissue samples, without having to use any chemical modification, ideally in vivo, giving outstanding significance to these methods in the field of cancer research. The first ambient MS method described was desorption electrospray ionization mass spectrometry (DESI-MS), which was implemented by directing a pneumatically assisted solvent electrospray onto the surface of interest. Herein, DESI-MS can be utilized to detect metabolomic changes in the mouse models, and to uncover the mechanism by which exemplary azapodophyllotoxin derivatives disrupt cancer progression metabolically.

### Screening Methods

Aspects of the present disclosure also include assays configured to identify agents that find use in methods of the invention, e.g., as reviewed above. Aspects of the present disclosure include methods for identifying a candidate compound for suppressing cancer. In some instances, the method comprises: contacting a cancer cell with a candidate compound; determining if tubulin protein levels are decreased relative to the cancer cell in the absence of the candidate compound; and determining if monoglycerol levels are increased relative to the cancer cell in the absence of the candidate compound; wherein a decrease in tubulin protein level and an increase in levels of monoglycerols identifies the candidate compound as a cancer suppressing compound. In the subject methods, a decrease in tubulin protein is indicative of inhibition of tubulin polymerization; and an increase in monoglycerol levels is indicative of inhibition of monoglycerol metabolism.

By assessing or determining is meant at least predicting that a given test compound will have a desirable activity, such that further testing of the compound in additional assays, such as animal model and/or clinical assays, is desired.

The candidate agent is selected from: a small molecule, an oligonucleotide, an antibody and a polypeptide. In some instances, the determining step comprises detecting a cellular parameter, wherein a change in the parameter in the cell as compared to in a cell not contacted with candidate agent indicates that the candidate agent specifically inhibits tubulin polymerization and monoglycerol metabolism. In certain cases, the tubulin protein levels are determined by a nano-fluidic proteomic immunoassay (NIA). In certain cases, the monoglycerol levels are determined by desorption electrospray ionization mass spectrometry imaging (DESI-MSI). In certain instances, the candidate agent is an azapodophyllotoxin derivative. In some instances the cancer is a renal cancer. In some instances, the cancer is lymphoma.

Drug screening may be performed using an in vitro model, a genetically altered cell or an animal. One can identify ligands that compete with, modulate or mimic the action of a lead agent. Drug screening identifies agents that inhibit tubulin polymerization and monoglycerol metabolism. A wide variety of assays may be used for this purpose, including labeled in vitro binding assays, electrophoretic mobility shift assays, immunoassays for protein binding, nano-fluidic proteomic immunoassay, mass spectrometry methods and the like.

The term “candidate compound” as used herein describes any molecule, e.g., azapodophyllotoxin derivative, other small molecule, oligonucleotide, protein or pharmaceutical, with the capability of inhibit tubulin polymerization and monoglycerol metabolism. Generally, a plurality of assay mixtures are run in parallel with different agent concentrations to obtain a differential response to the various concentrations. Typically one of these concentrations serves as a negative control, i.e., at zero concentration or below the level of detection.

Candidate compounds encompass numerous chemical classes, such as oligonucleotides, antibodies, polypeptides, and organic molecules, e.g., small organic compounds having a molecular weight of more than 50 and less than about 2,500 Daltons. Candidate compounds comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Of interest in certain embodiments are compounds that pass the blood-brain barrier.

Where the screening assay is a binding assay, one or more of the molecules may be joined to a member of a signal producing system, e.g., a label, where the label can directly or indirectly provide a detectable signal. Various labels include, but are not limited to: radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

In some embodiments, the screening step is performed at about 1 to about 1000 micromolar concentration of the compounds, such as about 10 to about 500 micromolar or about 10 to about 100 micromolar concentration. In some cases, a dose response curve is assessed for each of the compounds. In certain cases, the compounds are assessed for binding at a single concentration.

### Utility

The compounds and methods of the invention, e.g., as described herein, find use in a variety of applications. Applications of interest include, but are not limited to: research applications and therapeutic applications. Methods of the invention find use in a variety of different applications including any convenient application where inhibition of tubulin polymerization and monoglycerol metabolism is desired.

The subject compounds and methods find use in a variety of research applications. The subject compounds and methods may be used in the optimization of the bioavailability and metabolic stability of compounds.

The subject compounds and methods find use in a variety of therapeutic applications. Therapeutic applications of interest include those applications in cancer treatment. Of particular interest is treatment of renal cancer and lymphoma.

The subject methods may also find use in identifying candidate compounds for the treatment of cancer. In addition, the subject methods include methods of monitoring tumor regression in vivo.

### Pharmaceutical Compositions

The herein-discussed compounds can be formulated using any convenient excipients, reagents and methods. In certain embodiments, there is provided a pharmaceutical composition comprising a subject azapodophyllotoxin derivative and a pharmaceutically acceptable excipient. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the subject compound and a second active agent (e.g., as described herein), e.g. a small molecule, a chemotherapeutic, an antibody, an antibody fragment, an antibody-drug conjugate, an aptamer, or a protein, etc. are administered to individuals in a formulation (e.g., in the same or in separate formulations) with a pharmaceutically acceptable excipient(s). In some embodiments, the second active agent is a chemotherapeutic agent. In certain embodiments the chemotherapeutic agent is a taxane e.g. pacliataxel.

In another aspect of the present invention, a pharmaceutical composition is provided, comprising, or consisting essentially of, a compound of the present invention, or a pharmaceutically acceptable salt, isomer, tautomer or prodrug thereof, and further comprising one or more additional active agents of interest. Any convenient active agents can be utilized in the subject methods in conjunction with the subject compounds. In some instances, the additional agent is a chemotherapeutic agent. The subject compound and chemotherapeutic agent, as well as additional therapeutic agents as described herein for combination therapies, can be administered orally, subcutaneously, intramuscularly, intranasally, parenterally, or other route. The subject compound and second active agent (if present) may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ. In certain cases, the therapeutic agents can be administered intranasally. In some cases, the therapeutic agents can be administered intratumorally.

In some embodiments, the subject compound and a chemotherapeutic agent are administered to individuals in a formulation (e.g., in the same or in separate formulations) with a pharmaceutically acceptable excipient(s). The chemotherapeutic agents include, but are not limited to alkylating agents, nitrosoureas, antimetabolites, antitumor antibiotics, plant (vinca) alkaloids, and steroid hormones. Peptidic compounds can also be used. Suitable cancer chemotherapeutic agents include taxane and active analogs and derivatives thereof; dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

The subject compound and second chemotherapeutic agent, as well as additional therapeutic agents as described herein for combination therapies, can be administered orally, subcutaneously, intramuscularly, parenterally, or other route. The subject compound and second chemotherapeutic agent may be administered by the same route of administration or by different routes of administration. The therapeutic agents can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### EXAMPLES

The following examples are put forth so as to provide those of ordinary skill in the art with a complete disclosure and description of how to make and use embodiments of the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

### General Methods

### 1. Cell Culture Condition

Human T-cell acute lymphoblastic leukemias CCRF, DND-41, KOPT-K1, human B-cell Burkitt's lymphoma P493-6, and murine lymphoma 6780 were used in this study. Human and mouse lymphomas were all maintained in RPMI supplemented with 10% (vol/vol) FBS, 1% glutamine, 1% sodium pyruvate, 1% non-essential amino acids, and Antibiotic-Antimycotic. All cell culture reagents used were obtained from Gibco (Thermo Fisher Scientific Inc.).

In addition, murine renal cell carcinoma E28 cell line was used in this study. These cells were maintained in DMEM supplemented with 10% (vol/vol) FBS, 1% glutamine, 1% sodium pyruvate, 1% non-essential amino acids, and Antibiotic-Antimycotic.

### 2. Cell Counting

A 50 microliter volume of cells was taken from the culture medium and combined with an equal volume of 0.4% Trypan blue stain. Then, 10 microliters of this 1:1 mixture was pipetted onto a slide and placed into a hemocytometer for cell counting. The viable cell count was used for measuring cell proliferation in the sample.

### 3. In Vivo Transplantation

Human T-ALL cell line CCRF was transplanted subcutaneously into immunodeficient NOD-scid gamma mice (NSG), which lack mature B-cells, T-cells and natural killer cells. Prior to transplantation, CCRF was cultured in RPMI supplemented with 10% (vol/vol) FBS, 1% glutamine, 1% sodium pyruvate, 1% non-essential amino acids, and Antibiotic-Antimycotic. On the day of transplantation, cells were centrifuged at 800 rcf for 5 minutes, the media was aspirated, and cells were resuspended in PBS solution. This was then mixed in a 1:1 ratio with Matrigel (Corning and BD Biosciences) and transplanted to each mouse using 100 microliter injection subcutaneously. The tumor was monitored for growth up until the size of 12-15 mm, which was the size at which treatment would begin. The mice were continuously monitored for tumor size throughout the course of treatment, and euthanized if tumor size ever reached the 20 mm limit as recommended by APLAC guidelines.

### 4. Tubulin Polymerization

The Tubulin Polymerization Assay (EMD Millipore) was used to confer the mechanism of azapodophyllotoxin derivatives. The polymerization reaction was conducted in a 96-well plate. Paclitaxel was used as the non-tubulin-inhibiting negative control, and Nocodazole was used as the tubulin-inhibiting positive control. First, a solution of 99% pure bovine tubulin stock solution in 1× polymerization buffer (PB) containing 1 mM GTP was made. Second, a 1:9 mixture of each drug and control with PB-GTP solution was also prepared. In each well, 60 microliters of the first solution (99% tubulin solution in 1×PB and 1 mM GTP) was combined with 60 microliters of one of each of the prepared 1:9 drug to PB-GTP solutions. All solutions were prepared on ice prior to combining and placement into the spectrophotometer. Tubulin assembly was measured through turbidity variation (light scattering) in the spectrophotometer every 30 seconds at 350 nm (with shaking in between each reading) over the course of 90 minutes.

### 5. Nano-Fluidic Proteomic Immunoassay (NIA) Protocol

NIA was performed using the ProteinSimple Nanopro 1000 machine. The final protein concentration loaded into each nano-fluidic capillary of the machine was 0.1 ug/uL. The primary antibody for Tubulin (Cell Signaling Technologies) was diluted 1:100. The primary antibody for HSP70 (Santa Cruz Biosciences) was diluted 1:500. The secondary anti-rabbit and anti-mouse HRP-conjugates were both diluted 1:100. Chemiluminescence signal was recorded after the addition of the detection reagent, which consists of a 1:1 ratio of luminol and peroxide. Analysis of the chemiluminescence data from NIA was performed via the ProteinSimple Compass software.

### 6. FNA and Core Biopsy

Fine needle aspirations (FNAs) are defined to be biopsies performed via the aspiration of the tumor using a needle of 21 CCs or smaller. Core biopsies are any biopsies done using a needle larger than 21 CCs. Techniques for performing FNAs and core biopsies are similar. The needle tip is plunged into the tumor and retracted, in a fast and repetitive motion, while applying suction at the needle tip. This essentially utilizes the sharp sides of the needle tip to scrape off pieces of the tumor upon plunging the tip in, and during retraction, suction is used to draw the free pieces into the needle to be collected as specimens for analysis. This can be done at any time during the course of treatment, but was typically done at the midpoint of treatment, or before the tumors became too large and the mouse had to be euthanized.

### 7. DESI-MSI

DESI-MSI functions by directing a pneumatically assisted solvent electrospray onto the surface of interest (Takats, Wiseman, Gologan, & Cooks, 2004). The electrosprayed solvent droplets dissolve certain chemical constituents off the surface investigated and also induce the formation of secondary charged-droplets that take off from the surface. These secondary droplets already contain constituents of the sample and produce molecular ions of the analytes upon evaporation in the atmospheric pressure interface of the mass spectrometer. This way, electrospray-like mass spectrometric information is obtained on the sample, featuring multiply charged ions and solvent adducts among other electrospray-specific spectral features.

### Example 1: Compound Synthesis

Compounds may be prepared using any convenient method. For example, by similar methods to those described by Kumar et al. “Synthesis of novel functionalized azapodophyllotoxin derivatives in search of potent anti-tumor agents.” J. Heterocycl. Chem. 2010, 47, 1275-1282; and Bohlin et al., “Podophyllotoxin derivatives: drug discovery and development. Drug Discovery Today.” 1996, 1, 343-351; and those methods described by Malhotra et al. PCT application No. PCT/US2019/021840, filed Mar. 13, 2018, the disclosure of which is herein incorporated by reference in its entirety. Many general references providing commonly known chemical synthetic schemes and conditions useful for synthesizing the disclosed compounds are also available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). Reactions may be monitored by thin layer chromatography (TLC), LC/MS and reaction products characterized by LC/MS and 1H NMR. Intermediates and final products may be purified by silica gel chromatography or by HPLC.

Exemplary synthetic schemes, which can be adapted for the synthesis of subject compounds, is shown below:

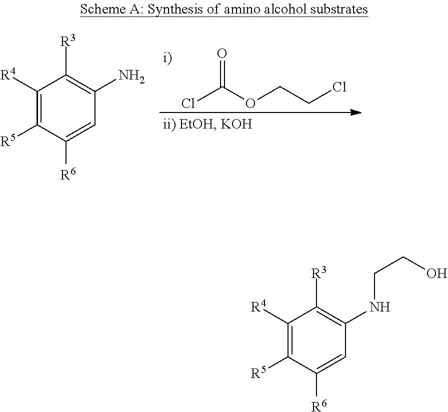

Amino alcohol substrates (i.e. which become Ring B of the subject compounds) were obtained by reacting corresponding commercially available aryl amines with chloroethylchloroformate, followed by reaction with potassium hydroxide in ethanol.

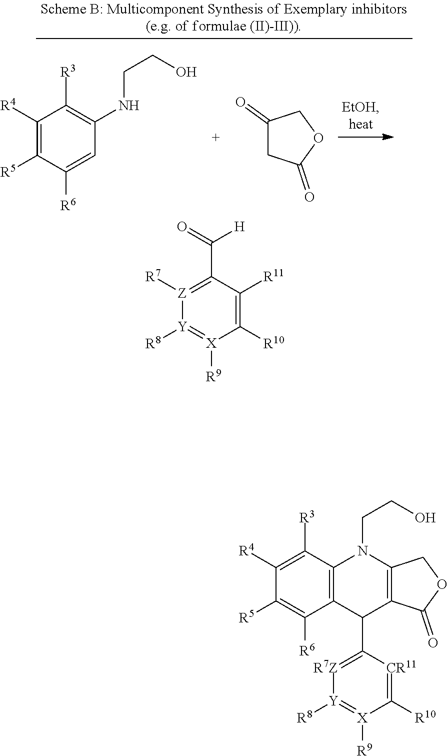

The amino alcohol substrates (e.g. as obtained from the reaction depicted in Scheme A above) were reacted with the corresponding commercially available benzaldehydes and tetronic acid using ethanol as a solvent to afford exemplary inhibitor compounds of formulae (II)-(III) as described herein.

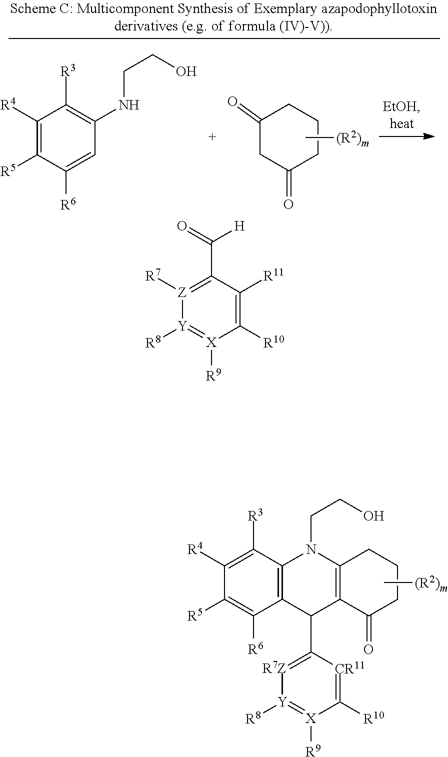

The amino alcohol substrates (e.g. as obtained from the reaction depicted in Scheme A above) were reacted with the corresponding commercially available benzaldehydes and corresponding 1,3-dione (e.g. 5,5-dimethylcyclohexane-1,3-dione or cyclohexane-1,3-dione) using ethanol as a solvent to afford exemplary inhibitor compounds of formulae (IV)-(V) as described herein.

### General Procedure for Synthesis of Exemplary Azapodophyllotoxin Derivatives (AZP) According to Scheme B:

An equimolar mixture of tetronic acid, substituted aniline, and aromatic aldehyde was dissolved in the minimum volume of ethanol. The reaction mixture was refluxed for 30-60 min. After cooling, the precipitate was filtered off, washed with minimal cold ethanol, and then recrystallized from ethanol to afford the desired inhibitor compound. (As previously reported: Tratrat, C.; Giorgi-Renault, S.; Husson, H. P., A multicomponent reaction for the one-pot synthesis of 4-aza-2,3-didehydropodophyllotoxin and derivatives. Organic. Lett. 2002, 19, 3187-3189).

Selected compounds as described herein and other derivatives were prepared and tested for activity in a variety of assays.

### Example 2: AZP Derivatives Suppresses Renal Cancer Cells (RCC) and Lymphoma Proliferation

Activity of compounds NSC750212, NSC750719, NSC750722, and NSC756089 (see, FIG. 1) was investigated on a MYC-driven RCC line derived from a transgenic mouse model using low dose ranges of 280 nM, 140 nM, and 70 nM. All these compounds showed very high sensitivity (FIG. 2). FIG. 2, illustrates the results of treatment of murine RCC E28 cells with, from left to right: DMSO and corresponding AZP derivatives at concentrations of 70 nM, 140 nM and 280 nM. Even at 70 nM, NSC750212 causes about 90% reduction of cell proliferation compared to the vehicle control (DMSO). At 140 nM, NSC750212 causes two-fold suppression of proliferation when compared to 70 nM, and any higher concentration (280 nM) shows no further efficacy. NSC756089 was least sensitive of all four compounds.

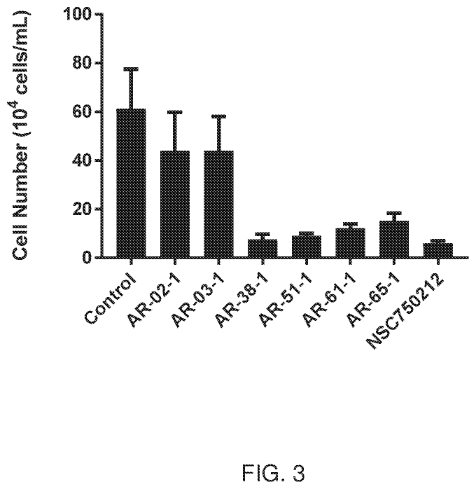

To further expand the structure activity relationship (SAR), six additional AZP derivatives were synthesized: AR-02, AR-03, AR-038, AR-051, AR-061 and AR-065, with different substituents on the C-4 phenyl ring (FIG. 1). Having validated that exemplary AZP compounds show efficacy in our murine-derived RCC line, it was sought to determine if this finding could be relevant in human RCC line, A498. Exemplary compound NSC750212 and newly synthesized analogues were tested in A498 cell line (FIG. 3). Upon testing at 70 nM, it was found that two compounds with no substitution on the C-4 phenyl ring, AR-02 and AR-03 show worse efficacy (30% reduction in viability) as opposed to NSC750212 (95% suppression). Compounds AR-061 and AR-065 show roughly 83% and 80% reduction in viability, respectively, which bring their potency closer to that of NSC750212. The most potent of the newly synthesized compounds are AR-038 and AR-051, both have single bromine substitution on the C-4 phenyl ring, with 90% and 87% suppression, respectively.

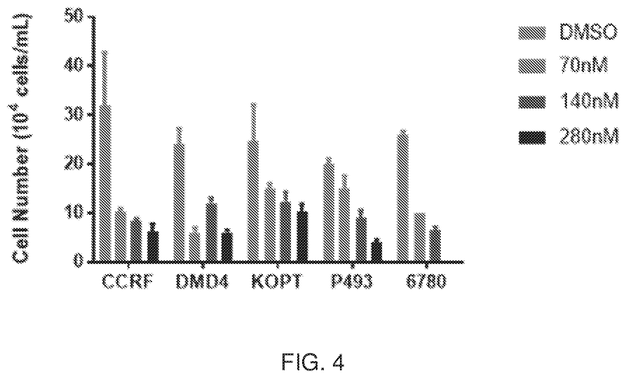

Activity of exemplary compounds against lymphomas was also investigated. It was observed that NSC750212 also shows dose-dependent suppression of proliferation in various human lymphoma lines of CCRF (T lymphoblast), DND-41 (child T lymphoblastic leukemia), and KOPT-K1 (human T-ALL), as well as in MYC-driven lymphoma lines (P4393, and 6780) (FIG. 4). FIG. 4 illustrates proliferative responses of various human lymphoma lines, from left to right: CCRF, DND-41, KOPR, and MYC-driven lymphoma lines P493 and 6780, at doses of 70 nM, 140 nM, and 280 nM vs a control (DMSO). At the 70 nM dose, CCRF and 6780 show an almost 90% reduction in proliferation, while DND-41 shows close to 95% reduction. KOPT and P-493 still show a significant but much less suppression, at roughly 85%. At 140 nM, CCRF and 6780 show an increase in suppression at roughly 92% and 94%, respectively. DND-41 shows no dose response, possibly due to 70 nM being the maximally effective dose in this cell line. KOPT shows a slight increase to roughly 87% suppression, whereas P-493 shows a larger increase to over 90% reduction in proliferation. Finally, at the 280 nM concentration, 6780 shows maximal suppression, and CCRF and P-493 show a continued increase in suppression to about 96% and 98%, respectively. KOPT has also increased to roughly 89% reduction of proliferation. Overall, NSC750212 demonstrates a dose-dependent response in CCRF, KOPT, P-493, and 6780, while reaching maximal dose in DND-41 at 70 nM.

### Example 3: AZP Derivative NSC750212 Causes RCC and Lymphoma Regression In Vivo

Having found the in vitro validation of NSC750212 efficacy in both lymphoma and RCC lines, it was sought to investigate if NSC750212 can impede cancer progression in vivo. Since NSC750212 shows highest efficacy in human RCC A498 line amongst the tested RCC lines (FIG. 2), and in human MYC-driven Burkitt Lymphoma line P493 amongst the tested lymphoma lines (FIG. 4), it was decided to transplant A498 and P493 into immunocompromised NSG mice for NSC750212 drug testing. Upon harvesting the tumors, it was discovered there to be a significant reduction in tumor burden in NSC750212 treated RCC (FIG. 5, panels A-C) and P493 (FIG. 6) after intratumoral treatment of NSC750212 for a week.

### Example 4: NSC750212 Inhibits Tubulin Polymerization

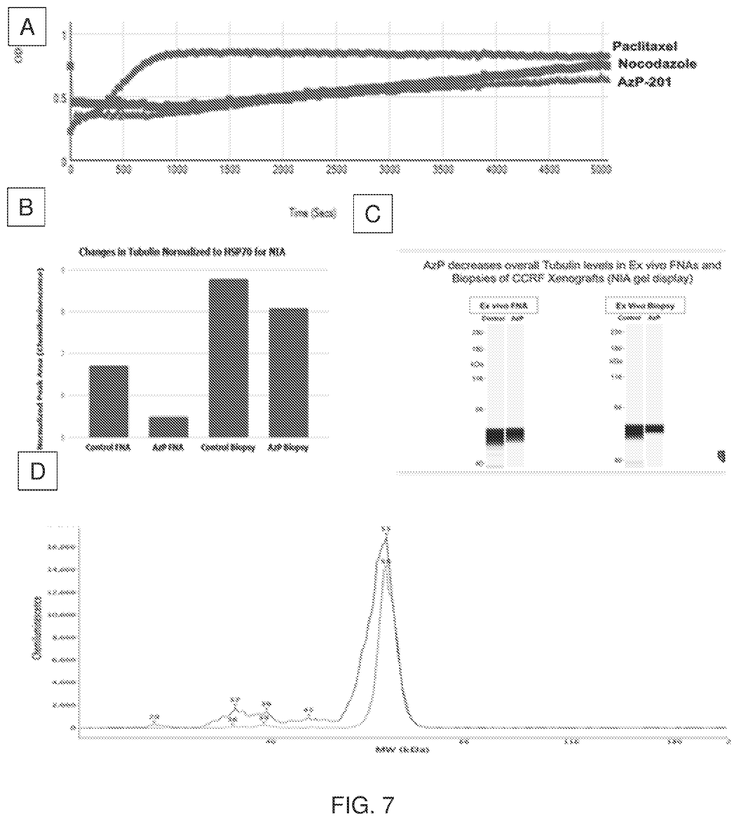

Given the structure of NSC750212 and computational docking studies of Azapodophyllotoxins in general, it has been stipulated that they may target tubulins. Using EMD Millipore Tubulin Polymerization Assay, NSC750212 was compared to a negative control, Paclitaxel, and a positive control, Nocodazole, to determine whether its mechanism is via the inhibition of tubulin polymerization. From this, our results indicate that NSC750212 can work through the inhibition of tubulin polymerization, such that it is more efficacious than Nocodazole in preventing depolymerization (FIG. 7, panel A).

To investigate if this mechanism is consistent in vivo, we used the Nano ImmunoAssay to analyze core biopsies and fine needle aspirates (FNA) of tumors during the course of treatment with NSC750212. After excising the tumor from the mice, ex vivo FNAs were also performed to conduct proteomic analysis on the tumors upon termination of treatment. These results are shown and seem to concur with previous results comparing tubulin levels in control and NSC750212 treated mice. Tumors treated with NSC750212 showed a decrease in tubulin up to termination of treatment. NSC750212 treated tumors showed lowered levels of tubulin expression in both the FNA and the core biopsy (FIG. 7, panel B) by at least two-fold. Size blot depiction of NIA also shows dramatic reduction of tubulin levels in NSC750212 treated group (FIG. 7, panel C). There are no other charged isoforms of tubulin detected by NIA (FIG. 7, panel D). This further indicates that NSC750212 could work via the inhibition of tubulin polymerization in human lymphoma CCRF Xenografts.

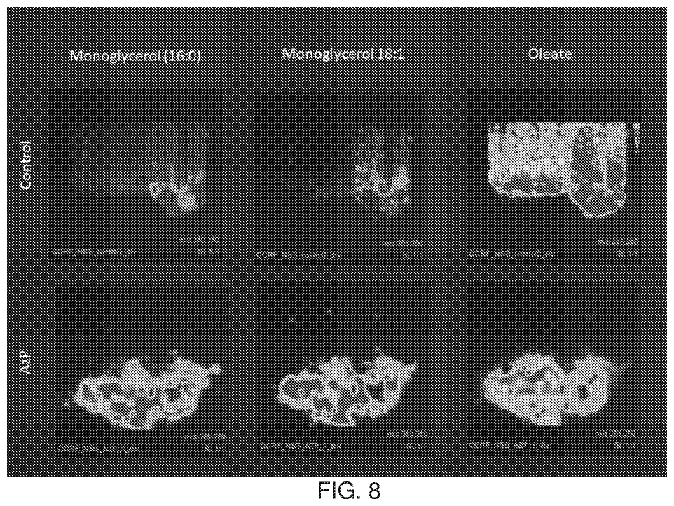

Though we expected a complete depletion of metabolism upon tubulin inhibition by NSC750212, our DESI-MSI analyses uncover a more interesting mechanism. RCC treated with NSC750212 shows elevated levels of monoglycerols compared to untreated RCC (FIG. 8). Monoglycerol metabolism has been implicated in cancer, due to the high need of cancer cells of energy and building blocks. The inhibition of monoglycerols have been shown to impede tumor growth. This metabolic effect of NSC750212 is surprising, and may explain why NSC750212 functions superior to that of conventional tubulin inhibitors. As described herein, DESI-MSI is a valuable tool for use in uncovering the mechanism of action of exemplary azapodophyllotoxin derivatives.

### DISCUSSION

Natural products have been a major source of pharmaceutical development due to their intrinsic biological relevance and often been referred as privileged structures that are likely to possess multiple biological activities. However, their complex structure and difficult synthesis impedes the hit-to-lead generation process of natural product-based drugs. One such example is podophyllotoxin (FIG. 1, compound 1), which is a potent but highly toxic tubulin polymerization inhibitor. Herein a single step multi-component reaction (MCR) was used to synthesize structurally simple aza-analogues of podophyllotoxin referred here as AZP derivatives or azapodophyllotoxin derivatives. The screening of exemplary AZP derivatives showed high efficacy in murine and human RCC cell lines E28 and A498, respectively (FIG. 2 and FIG. 3). SAR analysis suggest that compounds with unsubstituted phenyl ring were least active (AR-02 and AR-03) and the ones with tri-methoxy substitution on C-4 phenyl ring were most active (NSC750212). Compounds with halogenated substitution at C-4 phenyl ring also showed promising activity (AR-038 and AR-051). Compound NSC750212 also showed dose dependent inhibition in human lymphoma lines CCRF, DND-41, KOPT and MYC-driven lymphoma lines P493, and 6780 (FIG. 4).

The disclosure herein also demonstrates the importance of development of technologies to be able to assess drug mechanism of action as well as monitoring its therapeutic response using both NIA and DESI-MSI. NIA has been previously used to be able to detect the tissue origin of certain cancers, as well as the driving oncogene of the cancer from very small amount of sampling (Negi et al., Bioorganic & medicinal chemistry, 2015; 23(3):373-89; Steinmetz et al., Trends in cell biology, 2018; 28(10):776-92; Schiff et al. Nature, 1979; 277(5698):665-7). Here NIA has been further developed to be able to make those measurements from preclinical studies to allow time course measurements of tumors in vivo. Moreover, this method can then be used to monitor therapeutic response of a certain drug by measuring key biomarkers to assess the novel drug's potency not only at the study's endpoint. In addition to the mechanism of NSC750212 as a tubulin disruptor, DESI-MSI has uncovered that NSC750212 can also act to inhibit monoglycerol metabolism. The dual mode of action of NSC750212 provides a potential explanation as to the high potency of NSC750212.

Overall the above results demonstrate that the modification of natural compounds that are accompanied by nanoproteomic methods have been instrumental in developing lead compounds, such as NSC750212, a new tubulin and monoglycerol metabolic inhibitor for the treatment of cancers, such as RCC and lymphomas.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the following.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of inhibiting the proliferation of a cancer cell, the method comprising:
contacting the cell with an azapodophyllotoxin derivative;
wherein the contacting inhibits tubulin polymerization and monoglycerol metabolism to inhibit proliferation of cancer in the cell.

2. The method of claim 1, wherein the cancer cell is a renal cancer cell (RCC) or a lymphoma cell.

3. The method of claim 1 or 2, wherein the azapodophyllotoxin derivative is described by the formula (I): 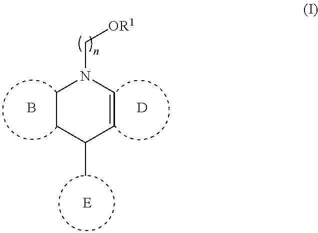
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
Ring B and Ring E are each independently selected from a C₅₋₆ membered carbocycle, a substituted C₅₋₆ membered carbocycle, a C₅₋₆ membered heteroaryl, a substituted C₅₋₆ membered heteroaryl, a C₅₋₆ membered heterocycle containing up to two atoms selected from N, O or S and a substituted C₅₋₆ membered heterocycle containing up to two atoms selected from N, O or S;
Ring D is selected from a C₅₋₆ carbocycle, a C₅₋₆ heterocycle containing up to two atoms selected from N, O or S, a substituted C₅₋₆ carbocycle, and a substituted C₅₋₆ heterocycle containing up to two atoms selected from N, O or S; and
n is an integer from 1 to 6,
or a pro-drug, a pharmaceutically acceptable salt or a solvate thereof.

4. The method of claim 3, wherein the Ring D is selected from: 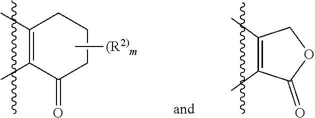
wherein:
each R² are independently selected from alkyl, substituted alkyl, acyl, substituted acyl, alkoxy, substituted alkoxy, carbocycle, substituted carbocycle, heterocycle, substituted heterocycle, heteroaryl, substituted heteroaryl, F, CF₃, CN, NO₂ and methoxy; and
m is an integer from 0 to 6.

5. The method of claim 3, wherein the Ring B or Ring E are each independently selected from aryl, substituted aryl, pyrrole, substituted pyrrole, imidazole, substituted imidazole, pyrazole, substituted pyrazole, furan, substituted furan, oxazole, substituted oxazole, isoxazole, substituted isoxazole, thiophene, substituted thiophene, thiazole, substituted thiazole, isothiazole, substituted isothiazole, pyridine, substituted pyridine, pyrimidine, substituted pyrimidine, 2-H-pyran, substituted 2-H-pyran, 2-H-thiopyran and substituted 2-H-thiopyran.

6. The method of claim 3, wherein the Ring B is of the formula (B1): 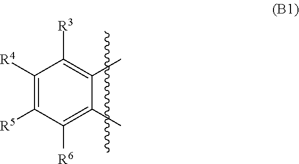
Wherein:
R³, R⁴, R⁵ and R⁶ are each independently selected from H, OH, methoxy, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S.

7. The method of claim 3, wherein the Ring E is of the formula (E1): 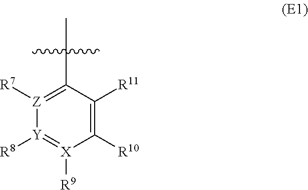
Wherein:
X, Y and Z are each independently selected from C or N; and
R⁷, R⁸, R⁹, R¹⁰ and R¹¹ are each independently selected from H, F, CF₃, CN, NO₂, Cl, Br, OH, alkyl and alkoxy;
or any of R⁷ and R⁸, R⁸ and R⁹, R⁹ and R¹⁰, R¹⁰ and R¹¹ together with the carbons to which they are attached form a C₅₋₆ carbocycle, C₅₋₆ heterocycle containing up to two atoms selected from N, O or S, a substituted C₅₋₆ carbocycle, or a substituted C₅₋₆ membered heterocycle containing up to two atoms selected from N, O or S.

8. The method of claim 3, wherein n is 2.

9. The method of claim 3, wherein the Ring B is selected from the formulae (B2) and (B3): 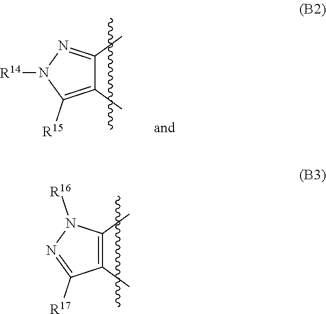
wherein:
R¹⁴, R¹⁵, R¹⁶ and R¹⁷ are each independently selected from H, alkyl, aryl and substituted aryl.

10. The method of claim 3, wherein the azapodophyllotoxin derivative is of the formula (II): 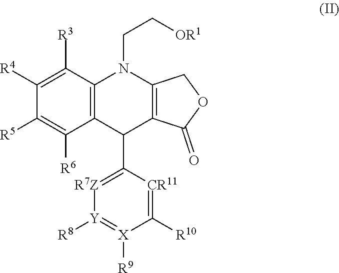
wherein: R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R³, R⁴, R⁵ and R⁶ are independently selected from H, OH, methoxy, alkyl, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, and a substituted C₅₋₆ membered heterocycle containing up to two atoms selected from N, O or S;
X, Y and Z are each independently selected from C or N;
R¹⁰ is selected from H, F, CF₃, CN, NO₂, OH, Cl, Br, methoxy and alkyl;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S,
or a pro-drug, a pharmaceutically acceptable salt or a solvate thereof.

11. The method of claim 3, wherein the azapodophyllotoxin is a structure selected from NSC750212, NSC750719, AR-02, AR-038, AR-061, NSC750722, NSC756089, AR-03, AR-051, and AR-065 (e.g., as shown in FIG. 1).

12. The method of claim 3, wherein the azapodophyllotoxin derivative is of the formula (III): 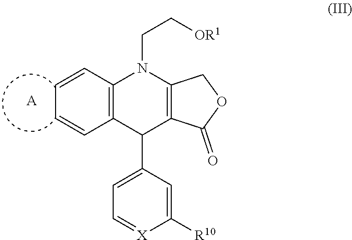
wherein:
Ring A is selected from a C₅₋₆ carbocycle, a C₅₋₆ heterocycle containing up to two atoms selected from N, O or S, a substituted C₅₋₆ carbocycle, and a substituted C₅₋₆ heterocycle containing up to two atoms selected from N, O or S;
X is C or N;
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R¹⁰ is selected from F, Br, CF₃, CN, NO₂, OH, alkyl and methoxy,
or a pro-drug, a pharmaceutically acceptable salt or a solvate thereof.

13. The method of claim 12, wherein the Ring A is selected from 1,3-dioxolane, cyclopentane, cyclopentene, 1,4-dioxane, cyclohexane, cyclohexene.

14. The method of claim 13, wherein the Ring A is 1,3-dioxolane.

15. The method of claim 13, wherein R¹⁰ is selected from Br, CF₃, methoxy and Cl.

16. The method of claim 13, wherein R¹⁰ is Br.

17. The method of claim 13, wherein X is C.

18. The method of claim 3, wherein the azapodophyllotoxin derivative is of the formula (IV): 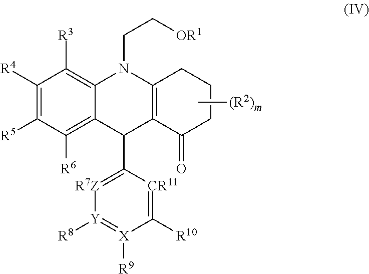
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R² is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R³, R⁴, R⁵ and R⁶ are independently selected from H, OH, methoxy, alkyl, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, and a substituted C₅₋₆ membered heterocycle containing up to two atoms selected from N, O or S;
X, Y and Z are each independently selected from C or N;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S; and
m is an integer from 0 to 6,
or a pro-drug, a pharmaceutically acceptable salt or a solvate thereof.

19. The method of claim 18, wherein the azapodophyllotoxin derivative is of the formula (V): 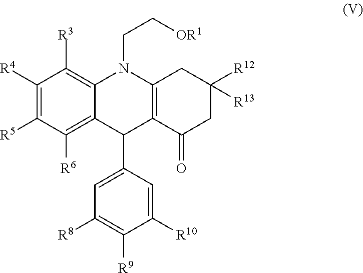
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R³, R⁴, R⁵ and R⁶ are independently selected from H, OH, methoxy, alkyl, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S;
R¹² and R¹³ are each independently selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl, F, CF₃, CN, NO₂ and methoxy;
R⁸, R⁹ and R¹⁰ are each independently selected from H, F, CF₃, CN, NO₂, methoxy, Cl, Br, OH and alkyl;
or any of R⁸ and R⁹ or R⁹ and R¹⁰ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S,
or a pro-drug, a pharmaceutically acceptable salt or a solvate thereof.

20. The method of claim 18 or 19, wherein R⁴ is methoxy and each of R³, R⁵ and R⁶ are H.

21. The method of claim 18 or 19, wherein each of R³, R⁴ and R⁶ are H and R⁵ is methoxy.

22. The method of claim 18 or 19, wherein R⁴ and R⁵ together with the carbons to which they are attached form a group selected from 1,3-dioxolane, cyclopentane, cyclopentene, 1,4-dioxane, cyclohexane, cyclohexene; and
each of R³ and R⁶ are H.

23. The method of claim 22, wherein R¹ and R⁵ together with the carbons to which they are attached form 1,3-dioxolane; and
each of R³ and R⁶ are H.

24. The method of claim 18 or 19, wherein R¹⁰ is F and R⁸ and R⁹ are both hydrogen.

25. The method of claim 18 or 19, wherein R⁸, R⁹ and R¹⁰ and each hydrogen.

26. The method of claim 18 or 19, wherein R⁸, R⁹ and R¹⁰ are each methoxy.

27. The method of claim 3, wherein the azapodophyllotoxin derivative is of the formula (VI): 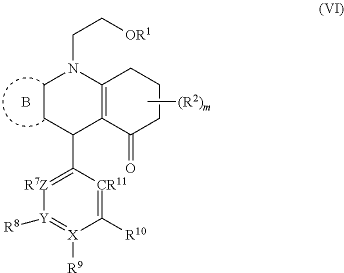
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R² is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
X, Y and Z are each independently selected from C or N;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S;
Ring B is selected from the formulae (B2) and (B3): 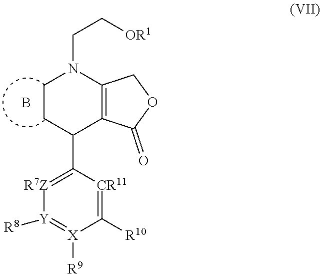
wherein R¹⁴, R¹⁵, R¹⁶ and R¹⁷ are each independently selected from H, alkyl, aryl and substituted aryl; and
m is an integer from 0 to 6,
or a pro-drug, a pharmaceutically acceptable salt or a solvate thereof.

28. The method of claim 3, wherein the azapodophyllotoxin derivative is of the formula (VII): 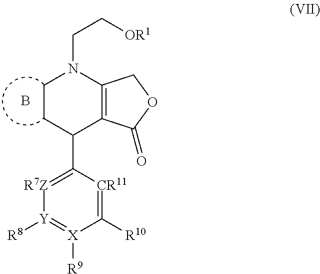
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
X, Y and Z are each independently selected from C or N;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S; and
Ring B is selected from the formulae (B2) and (B3): 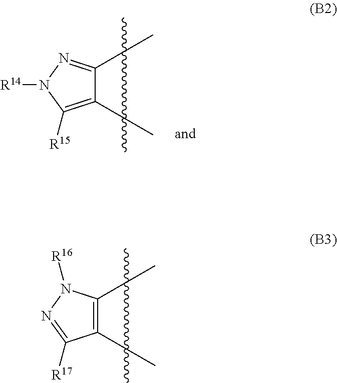
Wherein R¹⁴, R¹⁵, R¹⁶ and R¹⁷ are each independently selected from H, alkyl, aryl and substituted aryl;
or a pro-drug, a pharmaceutically acceptable salt or a solvate thereof.

29. The method of claim 27 or 28, wherein the Ring B is of the formula (B2).

30. The method of claim 27 or 28, wherein the Ring B is of the formula (B3).

31. A method of treating cancer, the method comprising:
administering to a subject in need thereof an effective amount of an azapodophyllotoxin derivative to treat the subject for cancer, wherein the cancer is selected from renal cancer and lymphoma.

32. The method of claim 31, wherein the administering is effective to inhibit tubulin polymerization and monoglycerol metabolism to treat the subject for cancer.

33. The method of claim 31 or 32, further comprising administration of one or more additional active agents.

34. The method of claim 37, wherein the one or more additional active agents is a small molecule, a chemotherapeutic, an antibody fragment, an antibody-drug conjugate, an aptamer, or a protein.

35. The method of claim 34, wherein the one or more additional agents is a chemotherapeutic agent.

36. The method of any one of claims 31-34, wherein the azapodophyllotoxin derivative is described by the formula (I): 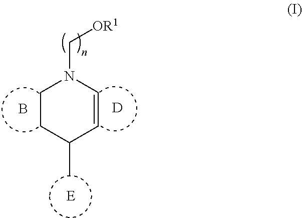
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
Ring B and Ring E are each independently selected from a C₅₋₆ membered carbocycle, a substituted C₅₋₆ membered carbocycle, a C₅₋₆ membered heteroaryl, a substituted C₅₋₆ membered heteroaryl, a C₅₋₆ membered heterocycle containing up to two atoms selected from N, O or S and a substituted C₅₋₆ membered heterocycle containing up to two atoms selected from N, O or S;
Ring D is selected from a C₅₋₆ carbocycle, a C₅₋₆ heterocycle containing up to two atoms selected from N, O or S, a substituted C₅₋₆ carbocycle, and a substituted C₅₋₆ heterocycle containing up to two atoms selected from N, O or S; and
n is an integer from 1 to 6,
or a pro-drug, a pharmaceutically acceptable salt or a solvate thereof.

37. The method of claim 36, wherein the Ring D is selected from: 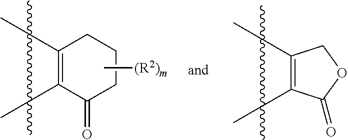
wherein:
each R² are independently selected from alkyl, substituted alkyl, acyl, substituted acyl, alkoxy, substituted alkoxy, carbocycle, substituted carbocycle, heterocycle, substituted heterocycle, heteroaryl, substituted heteroaryl, F, CF₃, CN, NO₂ and methoxy; and
m is an integer from 0 to 6.

38. The method of claim 36, wherein the Ring B or Ring E are each independently selected from aryl, substituted aryl, pyrrole, substituted pyrrole, imidazole, substituted imidazole, pyrazole, substituted pyrazole, furan, substituted furan, oxazole, substituted oxazole, isoxazole, substituted isoxazole, thiophene, substituted thiophene, thiazole, substituted thiazole, isothiazole, substituted isothiazole, pyridine, substituted pyridine, pyrimidine, substituted pyrimidine, 2-H-pyran, substituted 2-H-pyran, 2-H-thiopyran and substituted 2-H-thiopyran.

39. The method of claim 36, wherein the Ring B is of the formula (B1): 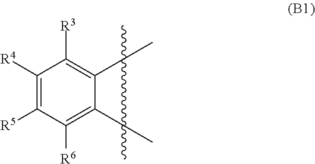
Wherein:
R³, R⁴, R⁵ and R⁶ are each independently selected from H, OH, methoxy, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S.

40. The method of claim 36, wherein the Ring E is of the formula (E1): 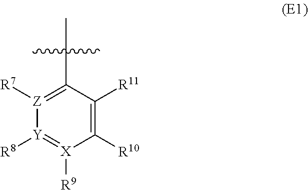
Wherein:
X, Y and Z are each independently selected from C or N; and
R⁷, R⁸, R⁹, R¹⁰ and R¹¹ are each independently selected from H, F, CF₃, CN, NO₂, Cl, Br, OH, alkyl and alkoxy;
or any of R⁷ and R⁸, R⁸ and R⁹, R⁹ and R¹⁰, R¹⁰ and R¹¹ together with the carbons to which they are attached form a C₅₋₆ carbocycle, C₅₋₆ heterocycle containing up to two atoms selected from N, O or S, a substituted C₅₋₆ carbocycle, or a substituted C₅₋₆ membered heterocycle containing up to two atoms selected from N, O or S.

41. The method of claim 36, wherein n is 2.

42. The method of claim 36, wherein the Ring B is selected from the formulae (B2) and (B3): 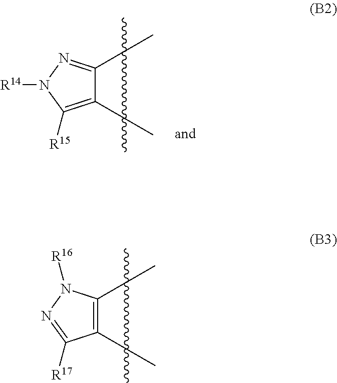
wherein:
R¹⁴, R¹⁵, R¹⁶ and R¹⁷ are each independently selected from H, alkyl, aryl and substituted aryl.

43. The method of claim 36, wherein the azapodophyllotoxin derivative is of the formula (II): 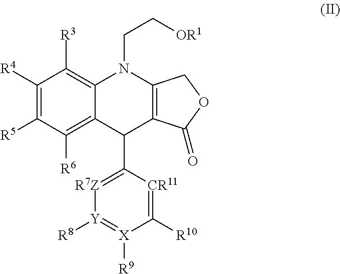
wherein: R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R³, R⁴, R⁵ and R⁶ are independently selected from H, OH, methoxy, alkyl, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, and a substituted C₅₋₆ membered heterocycle containing up to two atoms selected from N, O or S;
X, Y and Z are each independently selected from C or N;
R¹⁰ is selected from H, F, CF₃, CN, NO₂, OH, Cl, Br, methoxy and alkyl;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S,
or a pro-drug, a pharmaceutically acceptable salt or a solvate thereof.

44. The method of claim 36, wherein the azapodophyllotoxin is a structure selected from NSC750212, NSC750719, AR-02, AR-038, AR-061, NSC750722, NSC756089, AR-03, AR-051, and AR-065 (e.g., as shown in FIG. 1).

45. The method of claim 36, wherein the azapodophyllotoxin derivative is of the formula (III): 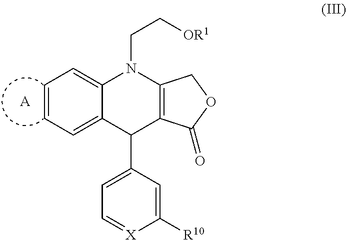
wherein:
Ring A is selected from a C₅₋₆ carbocycle, a C₅₋₆ heterocycle containing up to two atoms selected from N, O or S, a substituted C₅₋₆ carbocycle, and a substituted C₅₋₆ heterocycle containing up to two atoms selected from N, O or S;
X is C or N;
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R¹⁰ is selected from F, Br, CF₃, CN, NO₂, OH, alkyl and methoxy,
or a pro-drug, a pharmaceutically acceptable salt or a solvate thereof.

46. The method of claim 45, wherein the Ring A is selected from 1,3-dioxolane, cyclopentane, cyclopentene, 1,4-dioxane, cyclohexane, cyclohexene.

47. The method of claim 45, wherein the Ring A is 1,3-dioxolane.

48. The method of claim 45, wherein R¹⁰ is selected from Br, CF₃, methoxy and Cl.

49. The method of claim 45, wherein R¹⁰ is Br.

50. The method of claim 45, wherein X is C.

51. The method of claim 36, wherein the azapodophyllotoxin derivative is of the formula (IV): 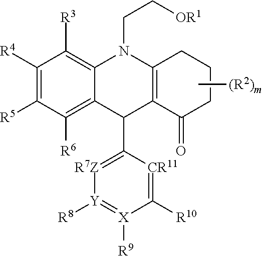
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R² is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R³, R⁴, R⁵ and R⁶ are independently selected from H, OH, methoxy, alkyl, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, and a substituted C₅₋₆ membered heterocycle containing up to two atoms selected from N, O or S;
X, Y and Z are each independently selected from C or N;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S; and
m is an integer from 0 to 6,
or a pro-drug, a pharmaceutically acceptable salt or a solvate thereof.

52. The method of claim 51, wherein the azapodophyllotoxin derivative is of the formula (V): 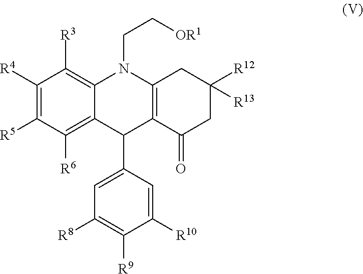
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R³, R⁴, R⁵ and R⁶ are independently selected from H, OH, methoxy, alkyl, halogen, CF₃, CN and NO₂;
or any of R⁴ and R⁵, R³ and R⁴, R⁵ and R⁶ together with the carbons to which they are attached form a C₅₋₆ carbocycle, a C₅₋₆ heterocycle containing up to two atoms selected from N, O or S, a substituted C₅₋₆ carbocycle, or a substituted C₅₋₆ membered heterocycle containing up to two atoms selected from N, O or S;
R¹² and R¹³ are each independently selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl, F, CF₃, CN, NO₂ and methoxy;
R⁸, R⁹ and R¹⁰ are each independently selected from H, F, CF₃, CN, NO₂, methoxy, Cl, Br, OH and alkyl;
or any of R⁸ and R⁹ or R⁹ and R¹⁰ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S,
or a pro-drug, a pharmaceutically acceptable salt or a solvate thereof.

53. The method of claim 51 or 52, wherein R⁴ is methoxy and each of R³, R⁵ and R⁶ are H.

54. The method of claim 51 or 52, wherein each of R³, R¹ and R⁶ are H and R⁵ is methoxy.

55. The method of claim 51 or 52, wherein R⁴ and R⁵ together with the carbons to which they are attached form a group selected from 1,3-dioxolane, cyclopentane, cyclopentene, 1,4-dioxane, cyclohexane, cyclohexene; and
each of R³ and R⁶ are H.

56. The method of claim 55, wherein R⁴ and R⁵ together with the carbons to which they are attached form 1,3-dioxolane; and
each of R³ and R⁶ are H.

57. The method of claim 51 or 52, wherein R¹⁰ is F and R⁸ and R⁹ are both hydrogen.

58. The method of claim 51 or 52, wherein R⁸, R⁹ and R¹⁰ and each hydrogen.

59. The method of claim 51 or 52, wherein R⁸, R⁹ and R¹⁰ are each methoxy.

60. The method of claim 36, wherein the azapodophyllotoxin derivative is of the formula (VI): 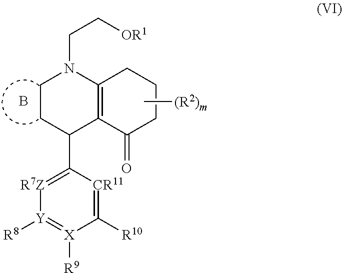
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
R² is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
X, Y and Z are each independently selected from C or N;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S;
Ring B is selected from the formulae (B2) and (B3): 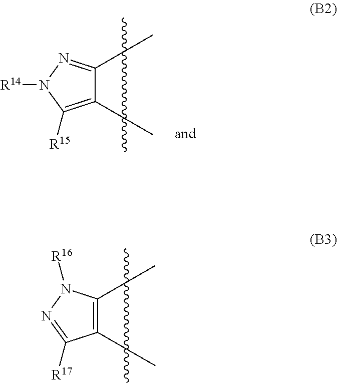
wherein R¹⁴, R¹⁵, R¹⁶ and R¹⁷ are each independently selected from H, alkyl, aryl and substituted aryl; and
m is an integer from 0 to 6,
or a pro-drug, a pharmaceutically acceptable salt or a solvate thereof.

61. The method of claim 36, wherein the azapodophyllotoxin derivative is of the formula (VII): 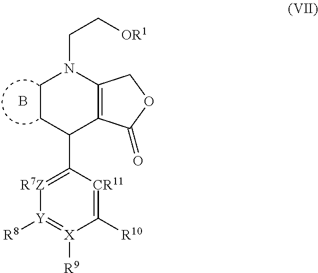
wherein:
R¹ is selected from H, alkyl, substituted alkyl, acyl, substituted acyl, alkoxy, substituted alkoxy, carbocycle, substituted carbocycle, heterocycle, substituted heterocycle, heteroaryl, substituted heteroaryl and a protecting group;
X, Y and Z are each independently selected from C or N;
R⁷, R⁸, R⁹, R¹⁰ and R¹¹ are each independently selected from H, F, CF₃, CN, NO₂, methoxy, Cl, Br, OH and alkyl;
or any of R⁷ and R⁸, R⁸ and R⁹, R⁹ and R¹⁰, R¹⁰ and R¹¹ together with the carbons to which they are attached form a C₅₋₆ carbocycle, or C₅₋₆ heterocycle containing up to two atoms selected from N, O or S, a substituted C₅₋₆ carbocycle, or a substituted C₅₋₆ membered heterocycle containing up to two atoms selected from N, O or S; and
Ring B is selected from the formulae (B2) and (B3): 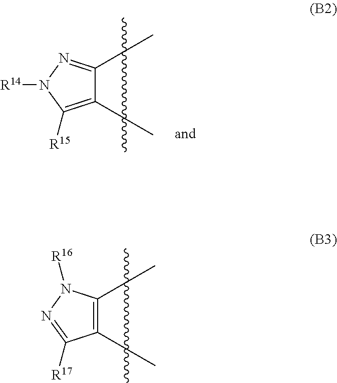
Wherein R¹⁴, R¹⁵, R¹⁶ and R¹⁷ are each independently selected from H, alkyl, aryl and substituted aryl;
or a pro-drug, a pharmaceutically acceptable salt or a solvate thereof.

62. The method of claim 60 or 61, wherein the Ring B is of the formula (B2).

63. The method of claim 60 or 61, wherein the Ring B is of the formula (B3).

64. A method of monitoring tumor regression in an individual, the method comprising:
assaying, in a sample obtained from the individual during a treatment regime for cancer, changes in tubulin protein levels, wherein a level of tubulin protein that is lower than a pre-treatment level of tubulin indicates tumor regression.

65. A method for identifying a cancer suppressing compound, the method comprising:
(a) contacting a cancer cell with a candidate compound;
(b) determining if tubulin protein levels are decreased relative to the cancer cell in the absence of the candidate compound, (where a decrease in tubulin protein level is indicative of inhibition of tubulin polymerization);
(c) determining if monoglycerol levels are increased relative to the cancer cell in the absence of the candidate compound, (where an increase in monoglycerol levels is indicative of inhibition of monoglycerol metabolism);
wherein a decrease in tubulin protein level and an increase in levels of monoglycerols identifies the candidate compound as a cancer suppressing compound.

66. The method of claim 65, wherein the tubulin protein levels is determined by a nano-fluidic proteomic immunoassay (NIA).

67. The method of claim 65 or 66, wherein the monoglycerol levels is determined by desorption electrospray ionization mass spectrometry imaging (DESI-MSI).

68. The method of claim any one of 65-67, wherein the cancer is selected from renal cancer or lymphoma.
